SEQUENCE LISTING (22 sequences)

SEQ ID NO: 1 - DNA, 21 bp, Artificial sequence
  Synthetic primer
accgcagctaggaataatgga

SEQ ID NO: 2 - DNA, 19 bp, Artificial sequence
  Synthetic primer
gcctcagttccgaaaacca

SEQ ID NO: 3 - DNA, 26 bp, Artificial sequence
  Synthetic primer
tgaacttagtgctgacttaaaggaac

SEQ ID NO: 4 - DNA, 22 bp, Artificial sequence
  Synthetic primer
acgaaaggagcctgagtttaag

SEQ ID NO: 5 - DNA, 23 bp, Artificial sequence
  Synthetic primer
ttaattatggtctgggttcttgg

SEQ ID NO: 6 - DNA, 20 bp, Artificial sequence
  Synthetic primer
aaggaagccagaaagcttcg

SEQ ID NO: 7 - DNA, 25 bp, Artificial sequence
  Synthetic primer
gcctgatacaaaatcaacattatca

SEQ ID NO: 8 - DNA, 21 bp, Artificial sequence
  Synthetic primer
gcagctgttcaggatgaaaac

SEQ ID NO: 9 - DNA, 22 bp, Artificial sequence
  Synthetic primer
cctgcgtattctactctcgatg

SEQ ID NO: 10 - DNA, 18 bp, Artificial sequence
  Synthetic primer
tacttaagccgcgccaag

SEQ ID NO: 11 - DNA, 21 bp, Artificial sequence
  Synthetic primer
gggaggagaagatgacctctg

SEQ ID NO: 12 - DNA, 19 bp, Artificial sequence
  Synthetic primer
cacatctgctctgctgtcg

SEQ ID NO: 13 - DNA, 21 bp, Artificial sequence
  Synthetic primer
cgggctagctcttcataatca

SEQ ID NO: 14 - DNA, 20 bp, Artificial sequence
  Synthetic primer
ccagcaactcgaagccatag

SEQ ID NO: 15 - DNA, 21 bp, Artificial sequence
  Synthetic primer
tccacaactgcacgatgtact

SEQ ID NO: 16 - DNA, 20 bp, Artificial sequence
  Synthetic primer
ccaggttcctcatcacctct

SEQ ID NO: 17 - DNA, 19 bp, Artificial sequence
  Synthetic primer
gcccctcatttgttttgga

SEQ ID NO: 18 - DNA, 20 bp, Artificial sequence
  Synthetic primer
ttcagcccatatcatgccta

SEQ ID NO: 19 - DNA, 20 bp, Artificial sequence
  Synthetic primer
cgttgaacatgtaggcagga

SEQ ID NO: 20 - DNA, 19 bp, Artificial sequence
  Synthetic primer
acttccaccatcgctacca

SEQ ID NO: 21 - DNA, 18 bp, Artificial sequence
  Synthetic primer
gccttgaacccctgatcc

SEQ ID NO: 22 - DNA, 22 bp, Artificial sequence
  Synthetic primer
ggtgggtattcatgatcgttct

(19) United States
(12) Patent Application Publication
(10) Pub. No.: US 2018/0147206 A1
(43) Pub. Date: May 31, 2018

(54) ACTIVATION OF NEURONAL STORE-OPERATED CALCIUM ENTRY PATHWAY FOR THE TREATMENT OF ALZHEIMER'S DISEASE

(71) Applicant: The Board of Regents of the University of Texas System, Austin, TX (US)

(72) Inventors: Ilya BEZPROZVANNY, Dallas, TX (US); Hua ZHANG, Plano, TX (US)

(73) Assignee: The Board of Regents of the University of Texas System, Austin, TX (US)

(21) Appl. No.: 15/572,292

(22) PCT Filed: May 4, 2016
(86) PCT No.: PCT/US16/30704

§ 371 Date: Nov. 7, 2017

Related U.S. Application Data
(60) Provisional application No. 62/159,083, filed on May 8, 2015.

(51) Int. Cl. A61K 31/517; A61P 25/28

(52) U.S. Cl. CPC A61K 31/517; A61P 25/28

(57) ABSTRACT

The present disclosure provides for new methods of treating Alzheimer's Disease. In particular, it concerns the activation of neuronal store-operated calcium entry pathway in Alzheimer's Disease patients.

### PRIORITY CLAIM

This application claims benefit of priority to U.S. Provisional Application Ser. No. 62/159,083, filed May 8, 2015, the entire contents of which are hereby incorporated by reference.

### STATEMENT OF FEDERAL FUNDING

This invention was made with government support under grant no. 1R01NS080152-01A1 awarded by the National Insitutes of Health. The government has certain rights in the invention.

### BACKGROUND

### I. Field

The present disclosure relates generally to the fields of neurobiology, neurophysiology, pharmacology and biochemistry. More particularly, it concerns the activation of neuronal store-operated calcium entry pathway in Alzheimer's Disease patients.

### II. Description of Related Art

Alzheimer's disease (AD) is the threat of modern humankind that is provoked by increased human lifespan. Despite extensive studies of AD pathology for more than 100 years, there are no disease-modifying therapies for AD. Memory loss in AD results from “synaptic failure” (Koffie et al., 2011; Selkoe et al., 2002 and Tu et al., 2014). Postsynaptic dendritic spines play an important role in learning and memory (Bourne et al., 2008 and Kasai et al., 2003). Postsynaptic spines are usually classified into 3 groups according to their morphological structure—mushroom spines, thin spines, and stubby spines (Bourne et al., 2008 and Kasai et al., 2003). It has been proposed that the mushroom spines are stable “memory spines” that make functionally stronger synapses which are responsible for memory storage (Bourne et al., 2007). The inventors and others previously proposed that mushroom spines are strongly eliminated in AD and that loss of mushroom spines may underlie cognitive decline during progression of the disease (Popugaeva et al., 2013; Popugaeva et al., 2012; Tackenberg et al., 2009 and Bezprozvanny et al., 2013). However, cell biological mechanisms responsible for loss of mushroom spines in AD are poorly understood.

Recently, the inventors demonstrated that neuronal store-operated calcium entry (nSOC) in postsynaptic spines play a key role in stability of mushroom spines by constitutively activating synaptic CaMKII (Sun et al., 2014). The inventors further demonstrated that synaptic nSOC is controlled by stromal interaction molecule 2 (STIM2) and that STIM2-nSOC-CaMKII pathway is compromised in PS1M146V knock-in (PS1KI) neurons, in aging neurons and in sporadic AD brains due to downregulation of STIM2 protein (Sun et al., 2014]. Moreover, the inventors have demonstrated that expression of STIM2 protein rescues synaptic nSOC and mushroom spine loss in PS1KI hippocampal neurons (Sun et al., 2014). In the follow up studies, they demonstrated that STIM2-nSOC pathway is downregulated in conditions of amyloid toxicity and that overexpression of STIM2 protects hippocampal mushroom spines from amyloid-induced loss (Popugaeva et al., 2015 and Zhang et al., 2015). These studies suggested that STIM2-nSOC pathway is a potentially important AD therapeutic target, however the molecular identity of STIM2-regulated nSOC channel in the synaptic spines is unknown.

### SUMMARY

Thus, in accordance with the present disclosure, there is provided a method of treating a mammalian subject with Alzheimer's Disease comprising administering to said subject a compound wherein the compound is further defined by the formula:

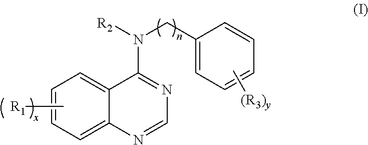

wherein:
each R₁ is independently selected from amino, cyano, carboxyl, halo, hydroxy, or nitro; or
alkylamino_{(C≤8)}, dialkylamino_{(C≤8)}, cycloalkylamino_{(C≤8)}, dicycloalkylamino_{(C≤8)}, or a substituted version of any of these groups;
x is 1, 2, 3, 4, or 5;
R₂ is hydrogen, alkyl_{(C≤8)}, or substituted alkyl_{(C≤8)};
n is 1, 2, 3, 4, or 5;
each R₃ is independently selected from amino, carboxyl, cyano, halo, hydroxy, or nitro; or
alkyl_{(C≤8)}, cycloalkyl_{(C<8)}, alkenyl_{(C≤8)}, alkynyl_{(C≤8)}, acyl_{(C≤8)}, amido_{(C≤8)}, or a substituted version of any of these groups; and
y is 1, 2, 3, 4, or 5;
or a pharmaceutically acceptable salt thereof. In some embodiments, the compound is further defined as:

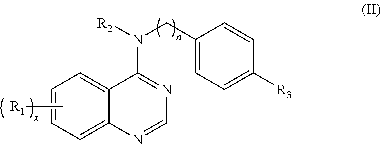

wherein: R₁, x, R₂, n, and R₃ are as defined above; or a pharmaceutically acceptable salt thereof. In some embodiments, the compound is further defined as:

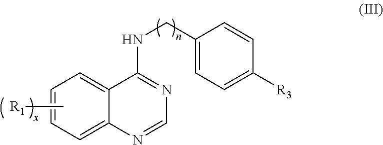

wherein: R₁, x, n, and R₃ are as defined above; or a pharmaceutically acceptable salt thereof. In some embodiments, the compound is further defined as:

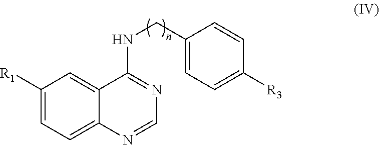

wherein: R₁, n, and R₃ are as defined above; or a pharmaceutically acceptable salt thereof. In some embodiments, R₁ is nitro. In other embodiments, R₁ is amino, alkylamino_{(C≤8)}, substituted alkylamino_{(C≤8)}, dialkylamino_{(C≤8)}, or substituted dialkylamino_{(C≤8)}. In some embodiments, n is 2 or 3. In some embodiments, n is 2. In some embodiments, R₃ is halo such as chloro. In other embodiments, R₃ is amido_{(C≤8)} or substituted amido_{(C≤8)} such as —NHC(O)CH₃. In some embodiments, the compound is further defined as:

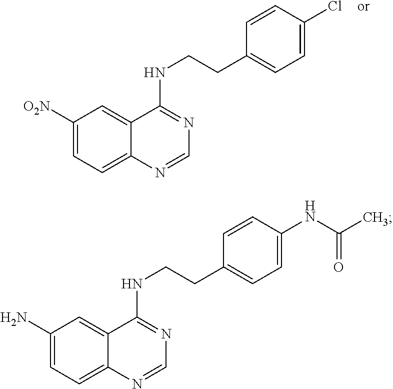 [SMILES: O=[N+]([O-])c1ccc2ncnc(NCCc3ccc(Cl)cc3)c2c1 ; CC(=O)Nc1ccc(CCNc2ncnc3ccc(N)cc23)cc1]

or a pharmaceutically acceptable salt thereof.

Also provided is a method of treating a mammalian subject with Alzheimer's Disease comprising administering to said subject an agonist or TRPC6 or Orai2, wherein said agonist is not hyperforin or a hyperforin derivative. Further provided is a method of treating a mammalian subject with Alzheimer's Disease comprising administering to said subject an agonist of the nSOC pathway, wherein said agonist is not hyperforin or a hyperforin derivative or analog. Even further provided is a method of treating a mammalian subject with Alzheimer's Dis comprising administering to said subject a potentiator of diacylglycerol (DAG)-induced TRPC6 activation.

The subject may be further treated with at least a second Alzheimer's Disease therapy, such as a cholinesterase inhibitor, a muscarinic agonist, an anti-oxidant, an anti-inflammatory, galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol or neuromidal. Treating may comprise one or more of improvements in memory, cognition or learning, slowing the progression of symptoms or pathophysiology, improving quality of life, or increasing life span. The compound or agonist may be administered orally or by injection, including intravenously, intradermally, intraarterially, intraperitoneally, intracranially, intraarticularly, intraprostaticaly, intrapleurally, intramuscularly, or subcutaneously. The compound or agonist may be administered 1, 2, 3 or 4 times daily. The compound or agonist may be administered chronically. The method may further comprise measuring cognition or memory in said subject prior to and/or after administration of said compound or agonist. The mammalian subject may be a human, such as one suffering from early onset Alzheimer's Disease or from late onset Alzheimer's Disease. The mammalian subject may be a non-human animal subject.

In still yet another embodiment, there is provided a pharmaceutical composition comprising a compound of the formula:

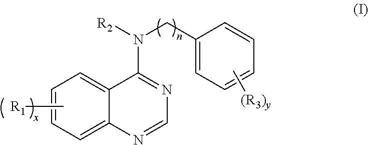

wherein:
each R₁ is independently selected from amino, cyano, carboxyl, halo, hydroxy, or nitro; or
alkylamino_{(C≤8)}, dialkylamino_{(C≤8)}, cycloalkylamino_{(C≤8)}, dicycloalkylamino_{(C≤8)}, or a substituted version of any of these groups;
x is 1, 2, 3, 4, or 5;
R₂ is hydrogen, alkyl_{(C<8)}, or substituted alkyl_{(C≤8)};
n is 1, 2, 3, 4, or 5;
each R₃ is independently selected from amino, carboxyl, cyano, halo, hydroxy, or nitro; or
alkyl_{(C≤8)}, cycloalkyl_{(C≤8)}, alkenyl_{(C≤8)}, alkynyl_{(C≤8)}, acyl_{(C≤8)}, amido_{(C≤8)}, or a substituted version of any of these groups; and
y is 1, 2, 3, 4, or 5;
   or a pharmaceutically acceptable salt thereof formulated in a pharmaceutical buffer, diluent or excipient. In some embodiments, the composition further comprises a compound further defined by the formula:

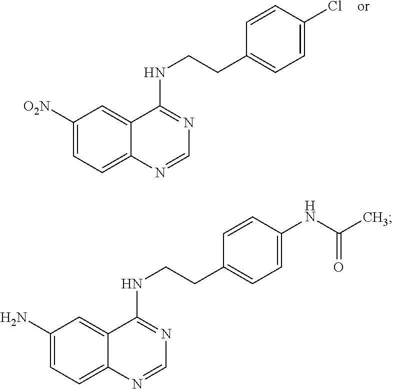 [SMILES: O=[N+]([O-])c1ccc2ncnc(NCCc3ccc(Cl)cc3)c2c1 ; CC(=O)Nc1ccc(CCNc2ncnc3ccc(N)cc23)cc1]

or a pharmaceutically acceptable salt thereof. The composition may be in a solid dosage form such as a tablet, a capsule or a powder. The composition may be in an oral liquid dosage form, or in an injectable liquid dosage form. The composition may comprise 1 to 100 mg/kg of said compound, 5-50 mg/kg or about 10 mg/kg, 15 mg/kg, 20 mg/kg, 25 mg/kg or 30 mg/kg.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the disclosure, and vice versa. Furthermore, compositions and kits of the disclosure can be used to achieve methods of the disclosure.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-E. TRPC6 and Orai2 form a STIM2 regulated complex in hippocampal synapse. (FIG. 1A) TRPC6 and Orai2 rabbit polyclonal antibodies were used to co-immunoprecipitate STIM2 from hippocampal synapsomal lysates. Input is 1/10 of lysate used for immunoprecipitation. (FIG. 1B) GST, GST-S2-SOAR and GST-S2-LASS recombinant proteins were used in pull-down experiments with lysates from HEK293 cells transfected with HA-Orai2 or YFP-TRPC6 expression constructs. The input is 1/50 of lysate used for pull down experiments. (FIG. 1C) Anti-HA mouse monoclonal antibodies or anti-TRPC6 rabbit polyclonal antibodies were used in immunoprecipitation experiments with lysates from HEK293 cells co-transfected with YFP-TRPC6 and HA-Orai2 expression plasmids. The input lane is 1/20 of lysate used for immunoprecipitation. (FIG. 1D) Anti-TRPC6 rabbit polyclonal antbodies were used in immunoprecipitation experiments with lysates from HEK293 cells co-transfected with HA-Orai2, HA-TRPC6 and YFP-STIM2 or YFP-STIM2-LASS constructs. Experiments 1 and 3 were performed with lysates prepared from cells incubated in normal ACSF (2 mM Ca²⁺) for 10 min prior to lysis. Experiments 2 and 4 were were performed with lysates prepared from cells incubated in Ca²⁺-free ACSF (with addition of 400 μM EGTA) for 10 min prior to lysis. The input lanes for experiments 1-4 contain 1/50 of total lysate used for immunoprecipitation. (FIG. 1E) Model that explains results shown on FIG. 1D (experiments 1-4). STIM2 binds to Orai2 directly and strongly via SOAR domain and weakly to TRPC6 via different region (experiment 1). Depletion of Ca²⁺ stores promotes assemembly of functional TRPC6/Orai2-STIM2 complex (experiment 2). STIM2-LASS mutant does not bind to Orai2 and instead recruited to non-productive complexes with TRPC6 (experiment 3). Association of STIM2-LASS mutant with Orai2 and TRPC6 is not affected by ER store depletion (experiment 4).

FIGS. 2A-J. TRPC6 and Orai2 are necessary for spine nSOC and mushroom spine maintenance. (FIGS. 2A-B) Western blot of analyis of lysates from wild-type hippocampal neurons cultures infected with lentiviruses encoding control RNAi (siCtrl), RNAi against TRPC6 (siT6, FIG. 2A) or RNAi against Orai2 (siO2, FIG. 2B). The lysates were blotted with antibodies agianst TRPC6, Orai2, PSD95, pCaMKII, and CAMKII as indicated. GAPDH was used as a loading control. Representative results from 3 independent cultures are shown. (FIGS. 2C and 2G) Time-course of GCaMP5.3 fluorescence signal changes in the spines of wild-type (FIG. 2C) and PS1KI (FIG. 2G) hippocampal neurons. The time of extracellular Ca²⁺ re-addition is indicated by a black bar above the traces. 100 μM DHPG was added 50 sec prior to Ca²⁺ re-addition. The neurons were infected with lentiviruses encoding control RNAi (siCtrl), RNAi against TRPC6 (siT6) or RNAi against Orai2 (siO2). The results for control neurons (Con) and neurons co-transfected with STIM2 expression plasmid (+STIM2) are shown. For each experimental group individual spine (grey) and average (red) fluorescence traces are shown. (FIGS. 2D and 2H) The average nSOC spine peak amplitude is shown for each group of cells shown on FIG. 2C (FIG. 2D) and FIG. 2G (FIG. 2H). The mean ΔF/F₀ signals for each group and presented as mean±SE (n≥45 spines). ***, p <0.001. FIGS. 2E and 2I. Confocal images of wild-type (FIG. 2E) and PS1KI (FIG. 2I) hippocampal neurons transfected with TD-Tomato at DIV7 and fixed at DIV15-16. The neurons were infected with lentiviruses encoding control RNAi (siCtrl), RNAi against TRPC6 (siT6) or RNAi against Orai2 (siO2). The results for control neurons (Con) and neurons co-transfected with STIM2 expression plasmid (+STIM2) are shown. Scale bar is 10 μm. (FIGS. 2F and 2J) The average fraction of mushroom spines for each group of cells shown on FIG. 2E (FIG. 2F) and FIG. 2I (FIG. 2J) is presented as mean±SE (n >20 neurons). ***, p <0.001, *, p <0.05.

FIGS. 3A-D. Functional roles of TRPC6 and Orai2 in supporting synaptic nSOC. (FIG. 3A) Time-course of GCaMP5.3 fluorescence signal changes in the spines of wild-type, PS1KI and APPKI hippocampal neurons. The time of extracellular Ca²⁺ re-addition is indicated by a black bar above the traces. 100 μM DHPG was added 50 sec prior to Ca²⁺ re-addition. The results are shown for control (CON) neurons and for neurons co-transfected with TRPC6, Orai2, STIM2, or STIM2-LASS plasmids as indicated. For each experimental group individual spine (grey) and average (red) fluorescence traces are shown. (FIG. 3B) The average nSOC spine peak amplitude is shown for each group of cells shown on FIG. 3A. The mean ΔF/F₀ signals for each group and presented as mean±SE (n≥73 spines). ***, p <0.001. (FIG. 3C) Confocal images of wild-type, PS1KI and APPKI hippocampal neurons transfected with TD-Tomato at DIV7 and fixed at DIV15-16. The images are shown for control neurons (CON) and for neurons co-transfected with TRPC6, STIM2, or STIM2-LASS plasmids. Scale bar is 10 μm. (FIG. 3D) The average fraction of mushroom spines for each group of cells shown on FIG. 3C is presented as mean±SE (n≥19 neurons). *** p <0.001.

FIGS. 4A-E. NSN21778 and Hyperforin rescue synaptic nSOC and mushroom spine loss in AD hippocampal neurons. (FIG. 4A) Chemical structure of NSN21778 and Hyperforin. (FIG. 4B) Time-course of GCaMP5.3 fluorescence signal changes in the spines of wild-type, PS1KI and APPKI hippocampal neurons. The time of extracellular Ca²⁺ re-addition is indicated by a black bar above the traces. 100 μM DHPG was added 50 sec prior to Ca²⁺ re-addition. The results are shown for control (CON) neurons and for neurons pretreated with 300 nM NSN21778 (+NSN) or 300 nM Hyperforin (+Hyp) for 30 min as indicated. For each experimental group individual spine (grey) and average (red) fluorescence traces are shown. (FIG. 4C) The average nSOC spine peak amplitude is shown for each group of cells shown on panel B. The mean AF/Fo signals for each group and presented as mean±SE (n≥45 spines). ***, p <0.001. (FIG. 4D) Confocal images of wild-type, PS1KI and APPKI hippocampal neurons transfected with TD-Tomato and fixed at DIV15-16. The images are shown for control neurons (CON) and for neurons treated with 30 nM NSN21778 (+NSN) or 30 nM Hyperforin (+Hyp) for 16 hours prior to fixation. Scale bar is 10 μm. (FIG. 4E) The average fraction of mushroom spines for each group of cells shown on FIG. 4D is presented as mean±SE (n≥18 neurons). ***, p <0.001.

FIGS. 5A-E. TRPC6 is a molecular target for NSN21778 and Hyperforin. (FIGS. 5A-B) Time course of Fura-2 Ca²⁺ signals is shown for HEK293 cells transfected with EGFP plasmid (GFP) or combination of EGFP and TRPC6 plasmids (TRPC6). Cells were incubated in ACSF media containing 2 mM Ca²⁺. In experiments shown in FIG. 5B, cells were moved to modified ACSF media containing 0.1 mM Ca²⁺ for 2 min and then returned to the media containing 2 mM Ca²⁺ with addition of 100 μM OAG. The time of addition of 1 μM Hyperforin (Hyp) or NSN21778 (NSN) is indicated by red bars above the Fura-2 traces. For each experimental group individual spine (grey) and average (red) fluorescence traces are shown. (FIG. 5C) The average Ca²⁺ influx peak is shown for experiments presented on FIGS. 5A-B as mean±SE (n≥81). (FIG. 5D) Confocal images of wild-type and PS1KI hippocampal neurons transfected with TD-Tomato and fixed at DIV15-16. The neurons were infected with lentiviruses encoding control RNAi (siCtrl), RNAi against TRPC6 (siT6) or RNAi against Orai2 (siO2). The images are shown for no drug treated neurons (CON) and for neurons treated with 30 nM NSN21778 (+NSN) or 30 nM Hyperforin (+Hyp) for 16 hours prior to fixation. Scale bar is 10 μm. No drug treated group of wild-type and PS1KI neurons is from the same experiment as shown on FIG. 2A-J. (FIG. 5E) The average fraction of mushroom spines for each group of cells shown on panel A is presented as mean±SE (n≥19 neurons). ***, p <0.001.

FIGS. 6A-E. NSN21778 rescues mushroom spine and synaptic plasticity defects in AD hippocampal slices. (FIG. 6A) Confocal images of CA1 hippocampal slices from 6 month-old WTGFP, PS1KIGFP, and APPKIGFP mice. The images are shown for untreated slices (CON) and for slices treated with 300 nM NSN21778 (+NSN) for 3.5 hrs prior to fixation. Scale bar is 10 μm. (FIG. 6B) The fraction of mushroom spines in hippocampal CA1 neurons from 6 month-old WTGFP, PS1KIGFP, and APPKIGFP mice. The results are shown for untreated slices (CON) and for slices treated with 300 nM NSN21778 (+NSN). The average fraction of mushroom spines for each group of cells is shown as mean±SE (n≥31 neurons). ***, p <0.001. (FIG. 6C) Sample fEPSP traces are shown for 6 month-old WT and APPKI hippocampal slices prior to stimulation (basal), immediately after tetanus stimulation (induction) and 1 h after tetanus stimulation (after 1 hr). The results are shown for untreated slices and for slices pre-treated with 300 nM NSN21778 (+NSN) for 2-3 hours prior to tetanus stimulation. (FIG. 6D) The normalized and averaged fEPSP slope is shown as a function of time in the experiments with 6 month-old wild-type and APPKI slices with (+NSN) or without 300 nM NSN21778 pre-treatment. At each time point the average normalized fEPSP slope is shown as mean±S.E (n≥6 mice). (FIG. 6E) The average normalized fEPSP slope 1 hour after tetanus stimulation is shown for 6 month-old wild-type and APPKI slices. The results are shown for untreated slices (CON) and for slices pre-treated 300 nM NSN21778 (+NSN) as mean±S.E. (n≥6 mice in each group). * p <0.05.

FIGS. 7A-G. NSN21778 rescues phenotypes of AD mice in vivo. (FIG. 7A) Confocal images of CA1 hippocampal slices from 6.5 month-old WTGFP, PS1KIGFP, and APPKIGFP mice. The images are shown for mice injected i.p. with vehicle solution (CON) and for mice injected with 10 mg/kg NSN21778 (+NSN) for 10 weeks. Scale bar is 10 μm. (FIG. 7B) The fraction of mushroom spines in hippocampal CA1 neurons from 6.5 month-old WTGFP, PS1KIGFP, and APPKIGFP mice. The results are shown for mice injected i.p. with vehicle solution (CON) and for mice injected with 10 mg/kg NSN21778 (+NSN) for 10 weeks. The average fraction of mushroom spines in each group is shown as mean±SE (n≥25 neurons). ***, p <0.001. (FIG. 7C) Images of coronal sections from 13 month-old APPKI mice stained with 6E10 anti-Aβ antibodies. The images are shown for or mice injected i.p. with vehicle solution (CON) and for mice injected with 10 mg/kg NSN21778 (+NSN) for 8 weeks. (FIGS. 7D-E) Average plaque area (FIG. 7D) and plaque intensity (FIG. 7E) is shown for slices from 13 month-old APPKI mice injected i.p. with vehicle solution (CON) and for mice injected with 10 mg/kg NSN21778 (+NSN) for 8 weeks. The data are shown as mean±SE (n=5 mice for NSN group, n=4 mice for CON group). *, p <0.05. The average fraction of time spent in the freezing state following contextual fear conditioning paradigm is shown for 6.5 month-old WTGFP and APPKIGFP mice injected i.p. with vehicle solution (CON) and for mice injected with 10 mg/kg NSN21778 (+NSN) for 10 weeks. The data are shown as mean±SE (n=5 mice). *, p <0.05. (FIG. 7F) The average fraction of time spent in the freezing state following contextual fear conditioning paradigm is shown for 6.5 month-old WTGFP and APPKIGFP mice injected i.p. with vehicle solution (CON) and for mice injected with 10 mg/kg NSN21778 (+NSN) for 10 weeks. The data are shown as mean±SE (n=5 mice). *, p <0.05. (FIG. 7G) STIM2-gated TRPC6/Orai2 nSOC channels play a critical role in maintenance of mushroom synpatic spines. Extracellular glutamate activates mGluR receptors in the spines, leading to activation of PLC, hydrolisis of PIP₂ and generation of InsP₃ and DAG. InsP₃ causes activation of InsP₃R1 in the ER Ca²⁺ stores in the spines, leading release of Ca²⁺ and depletion of the stores. Depletion of the stores causes oligomerization of STIM2 and activation of TRPC6/Orai2 Ca²⁺ influx channels. DAG generated following PIP2 hydrolysis acts as a co-factor in activating TRPC6/Orai2 channels. Resulting Ca²⁺ influx supports activity of CaMKII in the spines, which is necessary for long-term mushroom spine maintenance. NSN21778 compound (NSN) acts as a positive modulator of TRPC6/Orai2 channel activity, promoting Ca²⁺ influx in the spines and leading to rescue of mushroom spines, LTP and memory impairment in AD mouse models.

FIG. 8. TRPC and Orai channels Expression in Mouse Brain (Related to FIGS. 1A-E). In situ hybridization images from Allen Brain Atlas shows expression of STIMs, TRPCs and Orai channels in mouse brain.

FIG. 9. The gene expression profile of TRPC and Orai channels in different mouse brain regions (Related to FIGS. 1A-E). qRT-PCR results are presented as mean+SD of triplicate measurements for each gene transcript and brain region as indicated.

FIG. 10. Quantification for TRPC6, Orai2, PSD95, pCAMKII and CAMKII protein levels in in TRPC6 and Orai2 knockdown hippocampal neurons (Related to FIGS. 2A-J). Analysis of the data was performed using Quantity One software. The mean density of each band was normalized to GAPDH signal in the same sample and averaged. n=3 different batches cultures.

FIGS. 11A-B. Soma nSOC measurement in TRPC6 and Orai2 knock down hippocampal neuron cultures (Related to FIGS. 2A-J). (FIG. 11A) Time course of Fura-2 Ca²⁺ signals (F340/F380) is shown for WT hippocampal cultures infected with control lentiviruses and lentiviruses encoding siRNA against TRPC6 (siT6) or Orai2 (O2). Following 30 min store-depletion protocol, 2 mM Ca²⁺ aCSF was added back and Fura-2 signals in the soma were recorded. (FIG. 11B) The average SOC peak responses in soma of WT hippocampal neurons infected with Lenti-siCon, Lenti-siT6, or Lenti-si02 viruses are shown as mean±SE (n>59 neurons).

FIGS. 12A-D. NSN21778 in vitro metabolism stability, in vivo plasma pharmacokinetics, and side effect on body weight (Related to FIGS. 7A-G). (FIG. 12A) The metabolic stability of NSN21778 was evaluated in vitro after incubation with commercial liver S9 fractions in the presence of phase I (NADPH regenerating system) cofactors. Compound levels were determined by LC-MS/MS. Additionally, the stability of NSN21778 in the presence of commercial CD-1 mouse plasma was measured. (FIG. 12B) NSN21778 was dosed i.p. at 10 mg/kg to female CD-1 mice. Levels of NSN21778 in plasma and brain were evaluated by LC-MS/MS. (FIG. 12C) WTGFP, PS1KIGFP and APPKIGFP mice body weight after i.p. administrated with control solution or 10 mg/kg NSN21778 for 10 weeks. Data presented as mean±SE (n=5 mice). (FIG. 12D) Body weight gained for various treatments.

### DESCRIPTION OF ILLUSTRATIVE EMBODIMENTS

In the present study, the inventors used a candidate approach to demonstrate that STIM2-gated nSOC channels in the spines are formed by a complex of TRPC6 and Orai2. They further showed that known TRPC6 activator hyperforin (Leuner et al., 2007) and a novel nSOC activator NSN21778 can activate STIM2-nSOC pathway in the spines and rescue mushroom spine loss in PS1KI mice (Guo et al., 1999) and APPKI mice (Saito et al. 2014) hippocampal neurons. Furthermore, the inventors demonstrated that NSN21778 rescues hippocampal long-term potentiation (LTP) impairment, reduces amyloid burden and rescues hippocampal memory defects in APPKI mice. They concluded that STIM2-regulated TRPC6/Orai2 nSOC channel complex in dendritic mushroom spines is a new therapeutic target for treatment of memory loss in aging and AD and that NSN21778 is a potential candidate molecule for therapeutic intervention in brain aging and AD. These and other aspects of the disclosure are described in greater detail below.

### I. Alzheimer's Disease

Alzheimer's disease (AD) is a degenerative disorder of the brain first described by Alios Alzheimer in 1907 after examining one of his patients who suffered drastic reduction in cognitive abilities and had generalized dementia. It is the leading cause of dementia in elderly persons. AD patients have increased problems with memory loss and intellectual functions which progress to the point where they cannot function as normal individuals. With the loss of intellectual skills the patients exhibit personality changes, socially inappropriate actions and schizophrenia. AD is devastating for both victims and their families given that there is no effective palliative or preventive treatment for the inevitable neurodegeneration.

AD is associated with neuritic plaques measuring up to 200 μm in diameter in the cortex, hippocampus, subiculum, hippocampal gyrus, and amygdala. One of the principal constituents of neuritic plaques is amyloid, which is stained by Congo Red (Kelly et al., 1984)). Amyloid plaques stained by Congo Red are extracellular, pink or rust-colored in bright field, and birefringent in polarized light. The plaques are composed of polypeptide fibrils and are often present around blood vessels, reducing blood supply to various neurons in the brain.

Various factors such as genetic predisposition, infectious agents, toxins, metals, and head trauma have all been suggested as possible mechanisms of AD neuropathy. Available evidence strongly indicates that there are distinct types of genetic predispositions for AD. First, molecular analysis has provided evidence for mutations in the amyloid precursor protein (APP) gene in certain AD-stricken families (Goate et al., 1991; Murrell et al., 1991; Chartier-Harlin et al., 1991 and Mullan et al., 1992). Additional genes for dominant forms of early onset AD reside on chromosome 14 and chromosome 1 (Rogaev et al., 1995; Levy-Lahad et al., 1995 and Sherrington et al., 1995). Another loci associated with AD resides on chromosome 19 and encodes a variant form of apolipoprotein E (Corder, 1993).

Amyloid plaques are abundantly present in AD patients and in Down's Syndrome individuals surviving to the age of 40. The overexpression of APP in Down's Syndrome is recognized as a possible cause of the development of AD in Down's patients over thirty years of age (Rumble et al., 1989 and Mann et al., 1989). The plaques are also present in the normal aging brain, although at a lower number. These plaques are made up primarily of the amyloid β peptide (Aβ; sometimes also referred to in the literature as β-amyloid peptide or β peptide) (Glenner and Wong, 1984), which is also the primary protein constituent in cerebrovascular amyloid deposits. The amyloid is a filamentous material that is arranged in β-pleated sheets. AP is a hydrophobic peptide comprising up to 43 amino acids.

The determination of its amino acid sequence led to the cloning of the APP cDNA (Kang et al., 1987; Goldgaber et al., 1987; Robakis et al., 1987 and Tanzi et al., 1988) and genomic APP DNA (Lemaire et al., 1989 and Yoshikai et al., 1990). A number of forms of APP cDNA have been identified, including the three most abundant forms, APP695, APP751, and APP770. These forms arise from a single precursor RNA by alternate splicing. The gene spans more than 175 kb with 18 exons (Yoshikai et al., 1990). APP contains an extracellular domain, a transmembrane region and a cytoplasmic domain. Aβ consists of up to 28 amino acids just outside the hydrophobic transmembrane domain and up to 15 residues of this transmembrane domain. Aβ is normally found in brain and other tissues such as heart, kidney and spleen. However, Aβ deposits are usually found in abundance only in the brain.

Van Broeckhaven et al. (1990), have demonstrated that the APP gene is tightly linked to hereditary cerebral hemorrhage with amyloidosis (HCHWA-D) in two Dutch families. This was confirmed by the finding of a point mutation in the APP coding region in two Dutch patients (Levy et al., 1990). The mutation substituted a glutamine for glutamic acid at position 22 of the Aβ (position 618 of APP695, or position 693 of APP770). In addition, certain families are genetically predisposed to Alzheimer's disease, a condition referred to as familial Alzheimer's disease (FAD), through mutations resulting in an amino acid replacement at position 717 of the full length protein (Goate et al., 1991; Murrell et al., 1991 and Chartier-Harlin et al., 1991). These mutations co-segregate with the disease within the families and are absent in families with late-onset AD. This mutation at amino acid 717 increases the production of the Aβ₁₋₄₂ form of Aβ from APP (Suzuki et al., 1994). Another mutant form contains a change in amino acids at positions 670 and 671 of the full length protein (Mullan et al., 1992). This mutation to amino acids 670 and 671 increases the production of total Aβ from APP (Citron et al., 1992).

APP is processed in vivo at three sites. The evidence suggests that cleavage at the β-secretase site by a membrane associated metalloprotease is a physiological event. This site is located in APP 12 residues away from the lumenal surface of the plasma membrane. Cleavage of the β-secretase site (28 residues from the plasma membrane's lumenal surface) and the β-secretase site (in the transmembrane region) results in the 40/42-residue β-amyloid peptide (Aβ), whose elevated production and accumulation in the brain are the central events in the pathogenesis of Alzheimer's disease (for review, see Selkoe, 1999). Presenilin 1, another membrane protein found in human brain, controls the hydrolysis at the APP (β-secretase site and has been postulated to be itself the responsible protease (Wolfe et al., 1999). Presenilin 1 is expressed as a single chain molecule and its processing by a protease, presenilinase, is required to prevent it from rapid degradation (Thinakaran et al., 1996 and Podlisny et al., 1997). The identity of presenilinase is unknown. The in vivo processing of the β-secretase site is thought to be the rate-limiting step in Aβ production (Sinha & Lieberburg, 1999), and is therefore a strong therapeutic target.

The design of inhibitors effective in decreasing amyeloid plaque formation is dependent on the identification of the critical enzyme(s) in the cleavage of APP to yield the 42 amino acid peptide, the Aβ₁₋₄₂ form of Aβ. Although several enzymes have been identified, it has not been possible to produce active enzyme. Without active enzyme, one cannot confirm the substrate specificity, determine the subsite specificity, nor determine the kinetics or critical active site residues, all of which are essential for the design of inhibitors.

### II. Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

When used in the context of a chemical group: “hydrogen” means —H; “hydroxy” means —OH; “oxo” means ═O; “carbonyl” means —C(═O); “carboxy” means —C(═O)OH (also written as —COOH or CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means N═C═O; “azido” means —N₃; in a monovalent context “phosphate” means —OP(O)(OH)₂ or a deprotonated form thereof in a divalent context “phosphate” means —OP(O)(OH)O— or a deprotonated form thereof; “mercapto” means —SH; and “thio” means ═S; “sulfonyl” means —S(O)₂; “hydroxylsulfonyl” means —SO₂OH; “aminosulfonyl” means —SO₂NH₂ and “sulfinyl” means —S(O)—.

In the context of chemical formulas, the symbol “−” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

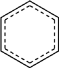

includes

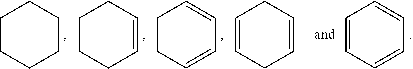

And it is understood that no one such ring atom forms part of more than one double bond. Furthermore, it is noted that the covalent bond symbol “−”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

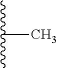

for methyl) mfficaies a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

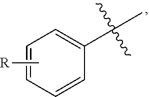

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

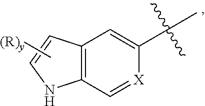

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and compound classes below, the number of carbon atoms in the group is as indicated as follows: “Cn” defines the exact number (n) of carbon atoms in the group/class. “C≤n” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. Also compare “phosphine_{(C≤10)}”, which designates phosphine groups having from 0 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefincs” are all synonymous. When any group or compound class below is used with the term “substituted”, any carbon atoms of the chemical group replacing the hydrogen atom do not count towards the total carbon atom limit for that group or compound class.

The term “saturated” when used to modify a compound or an atom means the compound or atom has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ^{l}Pr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tent-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂(methylene), —CH₂CH₂, —CH₂C(CH₃)₂CH₂, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound HR, wherein R is alkyl as this term is defined above. When any of these terms is used with the “substituted” modifier one or more hydrogen atom has been independently replaced by —OH, —F, —Cl, —Br, —I, —NH₂, —NO₂, CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

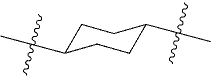 [SMILES: *C1CCC(*)CC1]

is a non-limiting example of cycloalkanediyl group. A “cycloalkane” refers to the compound H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the compound H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

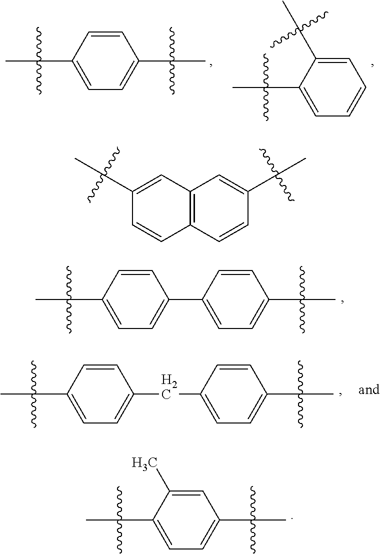

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group alkanediylaryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′ -methy lenebi s (3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2′, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

The compounds provided by the present disclosure are shown, for example, above in the summary of the invention section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds of the invention may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds of the invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### III. Neuronal Store-Operated Calcium Entry Pathway

A. SOCE Pathway

The existence of store-operated Ca²⁺ entry (SOCE) or capacitative Ca²⁺ entry was first proposed as a mechanism for Ca²⁺ influx regulated by the Ca²⁺ content of the intracellular pool rather than by the Ca²⁺ signals generated by agonists. In addition to the involvement of SOCE in Ca²⁺ store refilling, allowing cells to be ready for subsequent stimulus, SOCE has been reported to be important for a number of cellular functions, such as exocytosis, platelet function, muscle contraction, adenylyl cyclase activation, 5-lipoxygenase activation, endothelial permeability, gene transcription and ovulation and fertility.

In rat cortical astrocytes, SOCE maintain intracellular Ca²⁺ oscillations induced by stimulation of metabotropic glutamate receptors. Repetitive oscillations in [Ca²⁺¹]_{c} are spatio-temporal patterns of Ca²⁺ signals, recognized as physiological processes and that regulate a variety of cellular functions. A role for SOCE in Ca²⁺ oscillations was observed in rat hepatocytes, where treatment with 2-aminoethyl diphenylborate (2-APB), Gd³⁺ or SK&F 96365 inhibited vasopressin- and adrenaline-induced Ca²⁺ oscillations, High concentrations of Gd²⁺ prevent Ca²⁺ entry and extrusion, a condition that allowed Ca²⁺ oscillations to occur. SOCE sustains but is not essential for Ca²⁺ oscillations, since the mechanisms that initiate and regenerate Ca²⁺ oscillations are intrinsic to the intracellular medium.

SOCE has also been shown to be important for exocytosis in different cell types, including rat basophilic leukemia (RBL) cells and adrenal chromaffin cells. SOCE has also been suggested to be important for platelet function, and, on the base of the use of the sarco-endoplasmic reticulum Ca²⁺-ATPase inhibitors, such as thapsigargin (TG), SOCE has been shown to participate in muscle contraction, although TG does not provide unequivocal evidence for SOCE since it might induce opening of Ca²⁺-dependent Cl⁻ channels, resulting in depolarization and subsequent gating of voltage-activated Ca²⁺ channels.

SOCE is also required for the activation of certain enzymes. Ca²⁺ entry through store-operated Ca²⁺ channels can alter the activities of enzymes, such as the type I adenylyl cyclase in C6-2B glioma cells or the 5-lipooxygenase in RBL-1 cells. In addition, Ca²⁺ entry through store-operated channels regulates endothelial cell permeability. SOCE is also important for a number of long-term responses including gene transcription regulation. The physiological importance of SOCE is also supported by the identification of certain pathologies attributed to a failure or malfunction of this mechanism, including severe combined immunodeficiency (SCID), associated to a loss of SOCE in T-lymphocytes, or defective mast cell degranulation and cytokine secretion.

Neuroglial cells are homeostatic neural cells. Generally, they are electrically non-excitable and their activation is associated with the generation of complex intracellular Ca²⁺ signals that define the “Ca²+ excitability” of glia. In mammalian glial cells, the major source of Ca²⁺ for this excitability is the lumen of the endoplasmic reticulum (ER), which is ultimately (re)filled from the extracellular space. This occurs via store-operated Ca²⁺ entry (SOCE) which is supported by a specific signaling system connecting the ER with plasmalemmal Ca²⁺ entry. Here, emptying of the ER Ca²⁺ store is necessary and sufficient for the activation of SOCE, and without Ca²⁺ influx via SOCE the ER store cannot be refilled. The molecular arrangements underlying SOCE are relatively complex and include plasmalemmal channels, ER Ca²⁺ sensors, such as stromal interaction molecule, and possibly ER Ca²⁺ pumps (of the SERCA type). There are at least two sets of plasmalemmal channels mediating SOCE, the Ca²⁺-release activated channels, Orai, and transient receptor potential (TRP) channels. The molecular identity of neuroglial SOCE has not been yet identified unequivocally. However, it seems that Orai is predominantly expressed in microglia, whereas astrocytes and oligodendrocytes rely more on TRP channels to produce SOCE. In physiological conditions the SOCE pathway is instrumental for the sustained phase of the Ca²⁺ signal observed following stimulation of metabotropic receptors on glial cells. Two of these channels, TRPC6 and Orai2, are discussed below.

B. TRPC6

Transient receptor potential cation channel, subfamily C, member 6, also known as TRPC6, is a human gene encoding a protein of the same name. TRPC6 is a transient receptor potential ion channel. It has been associated with depression and anxiety (see below), as well as with focal segmental glomerulosclerosis (FSGS).

Two of the primary active constituents responsible for the antidepressant and anxiolytic benefits of Hypericum perforatum, also known as St. John's Wort, are hyperforin and adhyperforin. These compounds are inhibitors of the reuptake of serotonin, norepinephrine and epinephrine, dopamine, γ-aminobutyric acid, and glutamate, and they exert these effects by binding to and activating TRPC6. Activation of TRPC6 induces the entry of Ca²⁺ and Na⁺ into the cell, which results in the inhibition of reuptake. Although TRPC6 has been implicated in SOC in some studies, this channel is largely believed to be a receptor-operated channel (ROC), that can be directly activated by diacyl glycerol (DAG) (Estacion et al. 2004).

TRPC6 has been shown to interact with FYN, TRPC2 and TRPC3. The accession nos. are NM_004621 (mRNA) and NP_004612 (protein).

C. Orai2

Oria2 is a protein that in humans is encoded by the ORAI2 gene. Orai proteins, Orai1, Orai2 and Orai3 are STIM binding partners that form the pore of the channel. Orai proteins are uniformly distributed in the plasma membrane and exist as dimers in the resting state. STIM activation induces tetramerization of Orai proteins and subsequent STIM-Orai colocalization, which forms the active store-operated calcium channel. Orai2 functions as part of Ca²⁺ release-activated Ca²⁺-like (CRAC-like) channel subunit, which mediates Ca²⁺ influx, and increase in Ca²⁺-selective current by synergy with the Ca²⁺ sensor, STIM1.

Orai2 interacts with COPS6, GDF9, MED31, SETDB1 and UNC119. The accession nos. are NM_001126340 (mRNA) and NP_001119812 (protein).

### IV. Treatment of Alzheimer's Disease

A. Formulations and Routes of Administration

In accordance with the present disclosure, patients with Alzheimer's Disease are treated with the compounds described herein. It will be necessary to prepare pharmaceutical compositions in a form appropriate for administration to a subject. The compositions will generally be prepared essentially free of pyrogens, as well as other impurities that could be harmful to humans or animals. One will generally desire to employ appropriate salts and buffers to render stable cells suitable for introduction into a patient. Aqueous compositions of the present disclosure comprise an effective amount of stable cells dispersed in a pharmaceutically acceptable carrier or aqueous medium, and preferably encapsulated.

The phrase “pharmaceutically or pharmacologically acceptable” refer to compositions that do not produce adverse, allergic, or other untoward reactions when administered to an animal or a human. As used herein, “pharmaceutically acceptable carrier” includes any and all solvents, dispersion media, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the compositions of the present disclosure, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

The compositions will advantageously be administered orally or by injection, including intravenously, intradermally, intraarterially, intraperitoneally, intracranially, intraarticularly, intraprostaticaly, intrapleurally, intramuscularly, subcutaneously, or by other method or any combination of the forgoing as would be known to one of ordinary skill in the art.

As will be recognized by those in the field, a “therapeutically effective amount” refers to an mount of such that, when provided to a subject in accordance with the disclosed and claimed methods effectsone of the following biological activities: treatment of any aspect or symptom Alzheimer's Disease, including improvements in memory, cognition or learning, slowing the progression of symptoms or pathophysiology, improving quality of life, or increasing life span, in a subject diagnosed with or otherwise having Alzheimer's Disease.

As understood in the art, such therapeutically effective amount will vary with many factors including the age and weight of the patient, the patient's physical condition, the condition to be treated, and other factors. An effective amount of the disclosed compounds will also vary with the particular combination administered. However, typical doses may contain from a lower limit of about 1 μg, 5 μg, 10 μg, 50 μg to 100 μg to an upper limit of about 100 μg, 500 μg, 1 mg, 5 mg, 10 mg, 50 mg or 100 mg of the pharmaceutical compound per day. Also contemplated are other dose ranges such as 0.1 μg to 1 mg of the compound per dose. The doses per day may be delivered in discrete unit doses, provided continuously in a 24 hour period or any portion of that the 24 hours. The number of doses per day may be from 1 to about 4 per day, although it could be more. Continuous delivery can be in the form of continuous infusions. The terms “QID,” “TID,” “BID” and “QD” refer to administration 4, 3, 2 and 1 times per day, respectively. Exemplary doses and infusion rates include from 0.005 nmol/kg to about 20 nmol/kg per discrete dose or from about 0.01/pmol/kg/min to about 10 pmol/kg/min in a continuous infusion. These doses and infusions can be delivered by intravenous administration (i.v.) or subcutaneous administration (s.c.). Exemplary total dose/delivery of the pharmaceutical composition given i.v. may be about 2 μg to about 8 mg per day, whereas total dose/delivery of the pharmaceutical composition given s.c. may be about 6 μg to about 6 mg per day.

The disclosed compounds may be administered, for example, at a daily dosage of, for example: from about 0.01 mg/kg to about 100 mg/kg; from about 0.01 mg/kg to about 80 mg/kg; from about 0.01 mg/kg to about 70 mg/kg; from about 0.01 mg/kg to about 60 mg/kg; from about 0.01 mg/kg to about 50 mg/kg; from about 0.01 mg/kg to about 40 mg/kg; from about 0.01 mg/kg to about 30 mg/kg; from about 0.01 mg/kg to about 25 mg/kg; from about 0.01 mg/kg to about 20 mg/kg; from about 0.01 mg/kg to about 15 mg/kg; from about 0.01 mg/kg to about 10 mg/kg; from about 0.01 mg/kg to about 5 mg/kg; from about 0.01 mg/kg to about 3 mg/kg; from about 0.01 mg/kg to about 1 mg/kg; from about 0.01 mg/kg to about 0.3 mg/kg from about 100 mg/kg to about 90 mg/kg; from about 100 mg/kg to about 80 mg/kg; from about 100 mg/kg to about 70 mg/kg; from about 100 mg/kg to about 60 mg/kg; from about 100 mg/kg to about 50 mg/kg; from about 100 mg/kg to about 40 mg/kg; from about 85 mg/kg to about 10 mg/kg; from about 75 mg/kg to about 20 mg/kg; from about 65 mg/kg to about 30 mg/kg; from about 55 mg/kg to about 35 mg/kg; or from about 55 mg/kg to about 45 mg/kg. Administration may be by injection of a single dose or in divided doses.

The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

B. Combination Therapy

In another embodiment, the inhibitors of the present disclosure may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition includes an inhibitor of the present disclosure and the other includes the second agent(s).

The therapy of the present disclosure also may precede or follow the second agent treatment by intervals ranging from minutes to weeks. In embodiments where the other agent and the inhibitor of the present disclosure are administered separately, one may prefer that a significant period of time did not expire between the time of each delivery, such that the agent and present inhibitor would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, wherein the compound/agonists of the present disclosure disclosureis “A” and the secondary agent is “B”:

| | | | | |
|---|---|---|---|---|
| A/B/A | B/A/B | B/B/A | A/A/B | A/B/B |
| B/A/A | A/B/B/B | B/A/B/B | B/B/B/A | B/B/A/B |
| A/A/B/B | A/B/A/B | A/B/B/A | B/B/A/A | B/A/B/A |
| B/A/A/B | A/A/A/B | B/A/A/A | A/B/A/A | A/A/B/A |

It is expected that the treatment cycles would be repeated as necessary.

Various drugs for the treatment of AD are currently available as well as under study and regulatory consideration. The drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for AD.

### V. EXAMPLES

The following examples are included to demonstrate preferred embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Chemicals. NSN21778 (N-{4-[2-(6-Amino-quinazolin-4-ylamino)-ethyl]-phenyl}-acetamide) was synthesized and purified by Nanosyn Inc. (Santa Clara, Calif.).

Animals. The PS1-M146V knock-in mice (PS1KI) [Guo, 1999 #3782] were kindly provided by Hui Zheng (Baylor University). The APP^{NL-F} knock-in mice (APPKI) were kindly provided by Takaomi Saido (Riken, Japan) [Saito, 2014 #6473]. WT mice of the same strain (C57BL/6) were used in control experiments. PS1KIGFP and APPKIGFP mice were generated by crossing PS1KI or APPKI mice with line M GFP mice (C57BL/6 strain) [Feng, 2000 #6019]. All mice colonies were established and housed in a vivarium (4 per cage) with 12 h light/dark cycle at UT Southwestern Medical Center barrier facility. All procedures involving mice were approved by the Institutional Animal Care and Use Committee of the University of Texas Southwestern Medical Center at Dallas, in accord with the National Institutes of Health Guidelines for the Care and Use of Experimental Animals.

Dendritic Spine Analysis in Primary Hippocampal Neural Cultures. The hippocampal cultures of PS1KI, APPKI and WT mice were established from postnatal day 0-1 pups and maintained in culture as the inventors described previously [Zhang, 2010 #5733]. For assessment of synapse morphology, hippocampal cultures were transfected with TD-Tomato plasmid at DIV7 using the calcium phosphate method and fixed (4% formaldehyde, 4% sucrose in PBS, pH 7.4) at DIV15-16. A Z-stack of optical section was captured using 100× objective with a confocal microscope (Carl Zeiss Axiovert 100M with LSM510). 18-20 cultured neurons from three batches of cultures were used for quantitative analysis per genotype. Quantitative analysis for dendritic spines was performed by using NeuronStudio software package [Rodriguez, 2008 #6276]. To classify the shape of neuronal spines in culture, the inventors adapted an algorithm from published method [Rodriguez, 2008 #6276]. In classification of spine shapes the inventors used the following cutoff values: aspect ratio for thin spines (AR_thin_{(crio)})=2.5, head to neck ratio (HNR_{(crit)})=1.4, and head diameter (HD(crit))=0.5 μm. These values were defined and calculated exactly as described by [Rodriguez, 2008 #6276].

GCamp5.3 Ca²⁺ Imaging Experiments. GCamp5.3 imaging experiments were performed as the inventors previously reported [Sun, 2014 #6478]. Briefly, cultured hippocampal neurons were transfected with GCamp5.3 expression plasmid using calcium phosphate transfection method at DIV7. The GCamp5.3 fluorescent images were collected using Olympus IX70 inverted epifluorescence microscope equipped with a 60× lens, Cascade 650 digital camera (Roper Scientific) and Prior Lumen 200 illuminator. The experiments were controlled by the MetaFluor image acquisition software package (Universal Imaging). To measure synaptic nSOC, the neurons were moved from artificial CSF (aCSF) to calcium free media with 0.4 mM EGTA and 1 μM TG (thapsigargin) for 30 mins, after recording 30 sec basal, add 100 μM DHPG in calcium free aCSF, 50 sec later returned to aCSF with addition of Ca²⁺ channels inhibitor cocktail (1 μM TTX, 50 μM APS, 10 μM CNQX and 50 μM nifedipine). Analysis of the data was performed using NIH Image J software. The region of interest (ROI) used in the image analysis was chosen to correspond to spines. All Ca²⁺ imaging experiments were done in room temperature.

Hippocampal slice field recordings. Hippocampal slice field recordings were performed as recently described [Zhang, 2015 #6714]. Briefly, hippocampal slices (400 μm) were prepared from 6 month-old animals of either sex. Mice were anesthetized and transcardially perfused with dissection buffer before decapitation. The brain was removed, dissected, and sliced in ice-cold dissection buffer containing (in mM) 2.6 KCl, 1.25 NaH₂PO₄, 26 NaHCO₃, 0.5 CaCl₂, 5 MgCl₂, 212 sucrose, and 10 dextrose, using a vibratome (Leica VT 1000S). CA3 were cut off to avoid epileptogenic activity. The slices were transferred into a reservoir chamber filled with ACSF containing 124 mM NaCl, 5 mM KCl, 1.25 mM NaH₂PO₄, 26 mM NaHCO₃, 2 mM CaCl₂, 1 mM MgCl₂, and 10 mM dextrose. Slices were allowed to recover for 2-5 h at 30° C. ACSF and dissection buffer were equilibrated with 95% O₂-5% CO₂. For recording, slices were transferred to a submerged recording chamber, maintained at 30° C., and perfused continuously with ASCF at a rate of 2-3 ml/min. Field potentials (FPs) were recorded with extracellular recording electrodes (1 MΩ) filled with ACSF and placed in stratum radiatum of area CA1. FPs were evoked by monophasic stimulation (100-μs duration) of Schaffer collateral/commissural afferents with a concentric bipolar tungsten stimulating electrode (FHC, Bowdoinham, Me.). Stable baseline responses were collected every 30sec using a stimulation intensity (15-30 μA) yielding 50% of the maximal response. The initial slope of the FPs was used to measure stability of synaptic responses and quantify the magnitude of LTP. The LTP was induced by two trains of 100 Hz frequency stimulation for 1 sec, with each train separated by a 20 sec interval. For 14812 treatment experiments, hippocampal slices were pre-incubated with 300 nM 14812 for 2-3 hours prior to initiation of recordings in ACSF.

Dendritic Spine Analysis in Hippocampal slice. To analyze the shape of the spines in hippocampus slice the inventors used WTGFP, PS1KIGFP and APPKIGFP mice. Hippocampal slice were prepared as above, slices were allowed to recover for 1 h at 30° C., half slices were treated with 300 nM 14812 for 3.5 hours at 30° C., the other half slices stay in the ACSF as control, slices were fixed at 4% formaldehyde, 0.125% glutaraldehyde in PBS. GFP image were acquired by two-photon imaging (Zeiss LSM780) with 40× lens and 5× zoom. The Z interval was 0.5 μm. The secondary apical dendrites of hippocampal CA1 pyramidal neurons were selected for taking images. Approximately 25 neurons from 5 mice were analyzed for each genotype. To classify the shape of neuronal spines in slices the inventors also used NeuronStudio software package and an algorithm from [Rodriguez, 2008 #6276] with the following cutoff values: AR_thin_{(crit)}=2.5, HNR_{(crit)}=1.4, HD(crit)=0.5 μm.

NSN21778 in vivo study. For mushroom spine rescue and behavioral studies, 5 female mice for each group (WTGFP, PS1KIGFP and APPKIGFP) were i.p. injected 3 times/week with 10 mg/kg of NSN21778 starting at 4 months of age. Control groups of mice were injected with same solvent solution. After 6 weeks, injection routine was changed to 2 times per week. After 9 weeks, mice were tested by fear conditioning experiments. After 10 weeks, all mice were sacrificed for in vivo spine analysis. For amyloid plague study, APPKI mice were injected 10 mg/kg NSN21778 via i.p. injection 3 times/week starting at 11 months of age. Control group mice were inject with same solvent solution. After 8 weeks of injections, mice were sacrificed for AP immunohistochemistry staining.

Statistical Analyses. The results are presented as mean±SEM. Statistical comparisons of results obtained in experiments were performed by Student's t test for two-group comparisons and one-way or two-way ANOVA followed by Tukey test for multiple comparisons among more than two groups. The p values are indicated in the text and Figure legends as appropriate.

Plasmids and Viruses. YFP-STIM2 was kindly provided by Dr. Jen Liou, human TRPC6 cDNA and mouse Orai2 cDNA clones were purchased from Open Biosystems and used to generate TRPC6 and Orai2 lentiviral expression constructs by PCR, HA tag was induced to 5′end by PCR, YFP-TRPC6 was kindly provided by Dr. Craig Montell, FLAG-TRPC6/pCMV was kindly provided by Dr. Joseph Yuan, GST-S2-SOAR (aa 348-450) and GST-S2-CT (aa248-C terminal) was generated by PCR and cloned into PGEX-KG vector. STIM2-LASS (L377S, A380S) mutation was generated by Q5 mutagenesis Kit (Sigma), controlshort-hairpin RNA interference (Ctrl-shRNAi) (SHC002), mouse TRPC6shRNAi (SHCLNG-NM_013838, TRCN0000068394), and mouse Orai2-shRNAi (SHCLNG-NM_178751, TRCN0000126314) lentivirus shuttle constructs were obtained from Sigma. Lentiviruses were generated by co-transfection of two helper plasmids (pVSVg and pCMVΔ8.9) into the packaging cell line HEK293T as the inventors described previously (Zhang et al., 2010).

Antibodies. Anti-TRPC6 pAb (1:500, Sigma, SAB4300572), anti-Orai2 pAb (1:200 Santa Cruz, sc-292103), anti-GFP mAb(1:2000, Pierce, MA5-15256), anti-FLAG (1:1000, Sigma, F3165), anti-HA (1:3000, Covance, MMS-101R), anti-STIM2 pAb (1:500, Cell Signaling, 4917s), anti-Phospho-CaMKII (1:1000, Cell Signaling, 3361s), anti-CaMKII (1:1000, Chemicon, MAB8699), anti-PSD95 (1:1000, Cell Signaling, 3450s), anti-GAPDH (1:1000, Millipore, MAB374) , and Anti-A(3 6E10 mAb (1:1000, Covance, SIG-39300) were used. HRP-conjugated anti-rabbit and anti-mouse secondary antibodies (115-035-146 and 111-035-144) were from Jackson ImmunoReseach.

Quantitative reverse transcription PCR analysis (qRT-PCR). For mouse gene expression profiling, different brain region tissue were got from 7-8 weeks male C57BL/6 mice (n=6). RNA was extracted using RNAStat60 (TelTest, Friendswood, Tex.) according to the manufacturer's directions. Total RNA was pooled in equal quantities for each tissue (n=6). Genomic DNA contamination was eliminated by DNase I (Roche). cDNA for qPCR assays was prepared using High Capacity cDNA Reverse Transcription kit (Life Technologies). Gene expression levels were measured on an Applied Biosystems 7900HT with SYBR Green chemistry using the primers (see table below). Normalized mRNA levels are expressed as arbitrary units and were obtained by dividing the averaged, efficiency corrected values for mRNA expression by that for 18s rRNA (mouse 18s rRNA forward: accgcagctaggaataatgga; SEQ ID NO: 1, and mouse 18s rRNA reverse: gcctcagttccgaaaacca; SEQ ID NO: 2). The resulting values were multiplied by 10⁵ for graphical representation. Error bars represent experimental error and were calculated based on the standard deviations of the average value from triplicate sample wells.

| Gene | Forward | SEQ ID | Reverse | SEQ ID |
|---|---|---|---|---|
| Trpc1 | tgaacttagtgctgacttaaaggaac | 3 | cgggctagctcttcataatca | 13 |
| Trpc2 | acgaaaggagcctgagtttaag | 4 | ccagcaactcgaagccatag | 14 |
| Trpc3 | ttaattatggtctgggttcttgg | 5 | tccacaactgcacgatgtact | 15 |
| Trpc4 | aaggaagccagaaagcttcg | 6 | ccaggttcctcatcacctct | 16 |
| Trpc5 | gcctgatacaaaatcaacattatca | 7 | gcccctcatttgttttgga | 17 |
| Trpc6 | gcagctgttcaggatgaaaac | 8 | ttcagcccatatcatgccta | 18 |
| Trpc7 | cctgcgtattctactctcgatg | 9 | cgttgaacatgtaggcagga | 19 |
| Orai1 | tacttaagccgcgccaag | 10 | acttccaccatcgctacca | 20 |
| Orai2 | gggaggagaagatgacctctg | 11 | gccttgaacccctgatcc | 21 |
| Orai3 | cacatctgctctgctgtcg | 12 | ggtgggtattcatgatcgttct | 22 |

Hippocampal synaptosome fraction (P2) and co-immunoprecipitation. Hippocampal regions were extracted from 1 month old mice, homogenized in 0.32 M sucrose and 25 mM HEPES, pH 7.2, and centrifuged for 10 min at 800 g to remove the nuclei. The low-speed supernatant was then centrifuged for 20 min at 12,000 g to separate synaptosomal supernatant and synaptosomal membrane fractions (P2 pellet). P2 pellet were solubilized in lysis buffer containing 1% CHAPS, 137 mM NaCl, 2.7 mM KCl, 4.3 mM Na₂HPO₄, 1.4 mM KH₂PO₄, pH 7.2, 5 mM EDTA, 5 mM EGTA, and protease inhibitors for 2 hr at 4° C. Insoluble material was removed by centrifugation of samples for 20 min at 16300 g. Protein concentration in synaptosome fraction were measured by Nanodrop OD280. For each co-immunoprecipitation reaction, 500 μg total protein lysates were first pre-cleaned with normal rabbit IgG and Protein A/G beads at 4° C. for 1 hour, then incubate with 2 μg primary antibody at 4° C. for 1 hours, then incubate with 20 μl proteinA/G agarose beads at 4° C. overnight on a rocking platform, precipitated samples were then washed three times with lysis buffer, final beads pellet resuspended in 1×SDS loading buffer and analyzed by SDS-PAGE and Western Blot.

GST Pull-Down Assays. GST-fusion proteins were expressed in BL21 E. coli strain and purified as described previously (Zhang et al., 2005), YFP-TRPC6 or HA-Orai2 proteins were expressed in HEK293 cells and extracted in lysis buffer containing 1% CHAPS, 137 mM NaCl, 2.7 mM KCl, 4.3 mM Na₂HPO₄, 1.4 mM KH₂PO₄, pH 7.2, 5 mM EDTA, 5 mM EGTA, and protease inhibitors for 1 hr at 4° C. Extracts were clarified by centrifugation and incubated for 1 hr at 4° C. with the corresponding GST fusion protein. Beads were washed four times with the extraction buffer, and attached proteins were separated on SDS-polyacrylamide gel electrophoresis and probed with the anti-GFP or anti-HA antibody.

Fura-2 Ca²⁺ Imaging Experiments. Fura-2 Ca²⁺ imaging experiments with cultured DIV15-16 hippocampal neurons were performed as described previously (Zhang et al., 2010). Fura-2 340/380 ratio images were collected using a DeltaRAM-X illuminator, Evolve camera, and IMAGEMASTER PRO® software (all from Photon Technology International, Inc.). The entire cell somas were set as the region of interest (ROI) for image analysis. In neuronal store-operated Ca²⁺ entry (nSOC) experiments, the neurons were moved from artificial CSF (aCSF, 140 mM NaCl, 5 mM KCl, 1 mM MgCl₂, 2 mM CaCl₂, and 10 mM HEPES, pH 7.3) to calcium-free aCSF with addition of 0.4 mM EGTA and 1 μM Tg (thapsigargin) for 30 mins and then returned to aCSF with 1 μM TTX, 50 μM APS, 10 μM CNQX and 50 μM nifedipine. The maximal amplitude (peak) of nSOC-mediated Ca²⁺ increase was determined from Fura-2 340nm/380 nm ratio. All Ca²⁺ imaging experiments were performed at room temperature.

In vitro metabolism assays. Female ICR/CD-1 mouse S9 fractions were purchased from Celsis/In Vitro Technologies (Baltimore, Md.). 25 μl (0.5 mg) of S9 protein was added to a 15 ml glass screw cap tube. 350 μl of a 50 mM Tris, pH 7.5 solution, containing the compound of interest was added on ice. The final concentration of NSN21778 compound after addition of all reagents was 2 μM. 125 μl of an NADPH-regenerating system (1.7 mg/ml NADP, 7.8 mg/ml glucose-6-phosphate, 6 U/ml glucose-6-phosphate dehydrogenase in 2% w/v NaHCO₃/10 mm MgCl₂) was added for analysis of Phase I metabolism. The tube was then placed in a 37° C. shaking water bath. At varying time points after addition of phase I cofactors, the reaction was stopped by the addition of 0.5 ml of methanol containing formic acid and n-benzylbenzamide internal standard (IS.) The samples were incubated 10′ at RT and then spun at 16,100×g for 5 min. The supernatant was analyzed by LC-MS/MS. Stability in commercial mouse CD-1 plasma (Bioreclamation, Westbury, N.Y.) was measured in a similar fashion. NSN21778 (2 μM) was incubated with murine plasma for 0-1440 minutes. Reactions were quenched with methanol as above and supernatants evaluated by LC-MS/MS. Compound half-life was determined by the method of substrate depletion as described (McNaney et al., 2008).

In vivo pharmacokinetics. Six week-old female CD-1 mice were dosed IP (0.2 ml) with 10 mg/kg NSN21778 formulated in 10% ethanol/10% cremophor EL/80% 50 mM citrate buffer, pH5.0. Whole blood and brain was harvested. Acidified citrate dextrose (ACD) was used as the anticoagulant. Plasma was processed from whole blood by centrifugation for 10′ at 10,000 rpm in a standard centrifuge. Brains were weighed and snap frozen in liquid nitrogen. Brain homogenates were prepared by homogenizing the brain tissues in a 3-fold volume of PBS (total volume of homogenate in ml=4× weight in g). Total brain homogenate volume was estimated as volume of PBS added+volume of brain in ml. 100 μl of plasma or brain was mixed with 200 μl of acetonitrile containing formic acid to precipitate plasma or tissue protein and release bound drug. The samples were vortexed 15 sec, incubated at room temp for 10′ and spun 2×16,100 g. The supernatant was then analyzed by LC-MS/MS. Standard curves were prepared by addition of NSN21778 to plasma or brain homogenate. A value of 3× above the signal obtained in the blank plasma or brain homogenate was designated the limit of detection (LOD). The limit of quantitation (LOQ) was defined as the lowest concentration at which back calculation yielded a concentration within 20% of the theoretical value and above the LOD signal. The LOQ of NSN21778 for plasma and brain was 5 ng/ml. If values were above the LOD but below the LOQ, they were set to ½ LOQ or the actual measurement, whichever was highest. Because the animals were not perfused prior to tissue isolation, to determine the final brain concentration of NSN21778, the amount of compound in brain due to brain vasculature was first subtracted using the calculated plasma concentrations of NSN21778 and a value of 30 μl of blood/g of brain tissue (Kwon, 2001).

Fear conditioning test. Fear conditioning was measured in boxes equipped with a metal grid floor connected to a scrambled shock generator (Med Associates Inc., St. Albans). For training, mice were individually placed in the chamber. After 2 min, the mice received 3 tone-shock pairings (30 sec white noise, 80 dB tone co-terminated with a 2 sec, 0.5 mA footshock, 1 min intertrial interval). The following day, memory of the context was measured by placing the mice into the same chambers and freezing was measured automatically by the Med Associates software. Forty eight hours after training, memory for the white noise cue was measured by placing the mice in a box with altered floors and walls, different lighting, and a vanilla smell. Freezing was measured for 3 min, then the noise cue was turned on for an additional 3 min and freezing was measured.

Dendritic Spine Analysis in Mice Hippocampus. To analyze the shape of the spines in hippocampus in vivo, the inventors used GFP-M line mice (Feng et al., 2000) (WTGFP). To simplify the analysis, they crossed Line M GFP mice with PS1KI and APPKI mice to yield PS1KIGFP and APPKIGFP mice Mice were intracardially perfused with ice cold 4% paraformaldehyde (PFA) solution in phosphate buffer (pH 7.4) 30 ml in 3 min. The brains were extracted and post-fixed in 4% PFA solutions for 16 h before cutting. 50 μm hippocampal sections from the fixed brains were obtained using vibratome (Leica 1200S). A Z-stack of optical section was captured using 100× objective with a confocal microscope (Carl Zeiss Axiovert 100M with LSM510). The Z interval was 0.5 μm. The apical dendrites of hippocampal CA1 pyramidal neurons were selected for taking images. Approximately 25 neurons from 5 mice were analyzed for each group of mice. To classify the shape of neuronal spines in slices, the inventors also used NeuronStudio software package and an algorithm from (Rodriguez et al., 2008) with the following cutoff values: AR_thin_{(crit)}=2.5, HNR_{(crit)}=1.4, HD_{(crit)}=0.5 μm.

Aβ immunohistochemistry staining. For β-amyloid plaque analysis, mice were terminally anesthetized and perfused transcardially with 30 ml of ice-cold PBS, followed by 50 ml of fixative (4% paraformaldehyde in 0.1 M PBS, pH 7.4). All brains were removed from the skull, postfixed overnight at 4° C. in 4% paraformaldehyde, and equilibrated in 20-30% (w/v) sucrose in PBS. The brains were sliced to 30-μm-thick coronal sections using SM2000R sliding microtome (Leica). 30 μm coronal sections from APPPS1 mice spaced throughout the forebrain were stained with 6E10 anti-Aβ mAb (1:1000 dilution), followed by staining with Alexa Flour-488 secondary anti-mouse IgG (1:1000 dilution). The average area of the amyloid plaques and the average intensity of fluorescent signal in the plaques were calculated automatically for each slice by using Isocyte laser scanner and image analysis software as previously described (Zhang et al., 2010). Twenty coronal sections from each mice were quantified for the analysis, and the data were averaged within NSN21778 treated (n=5) and control groups (n=4).

### Example 2

### Results

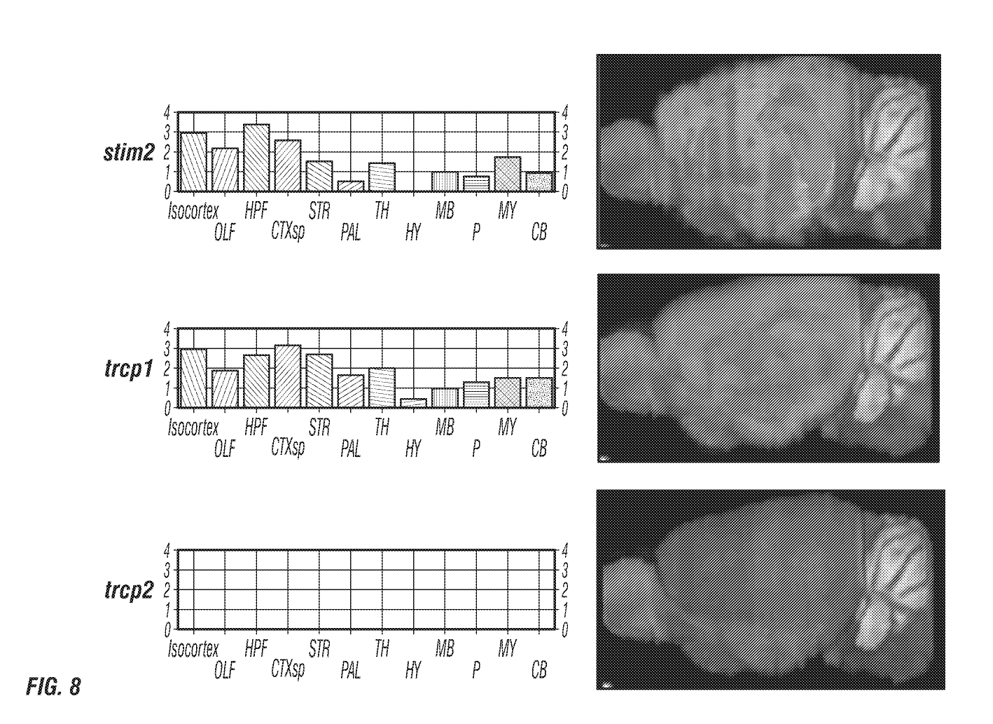

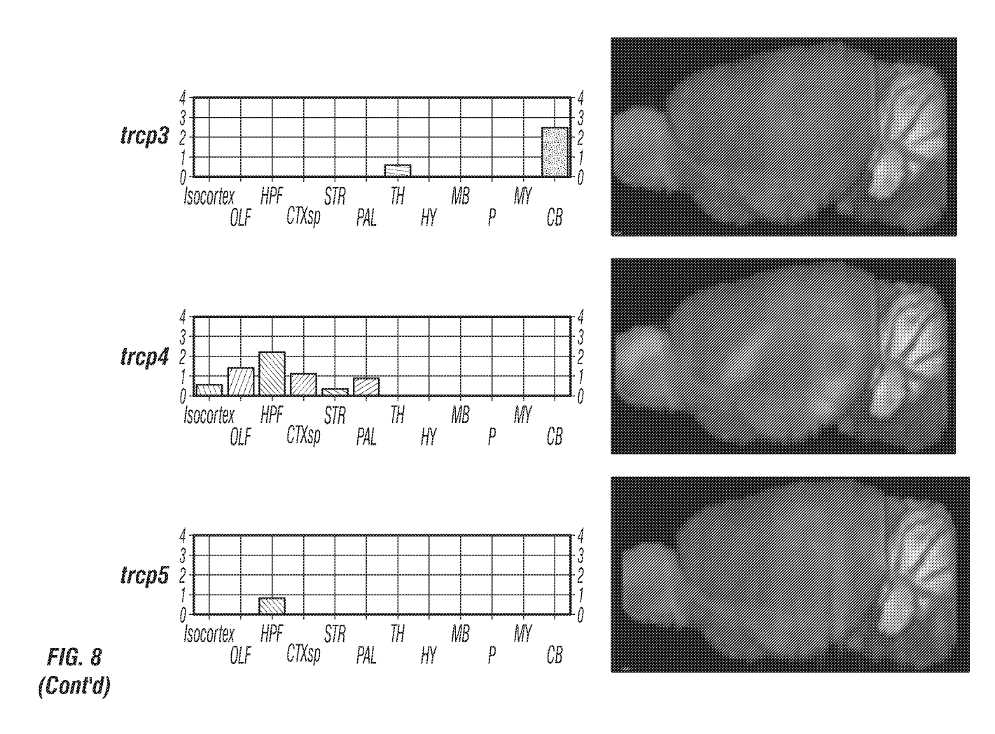

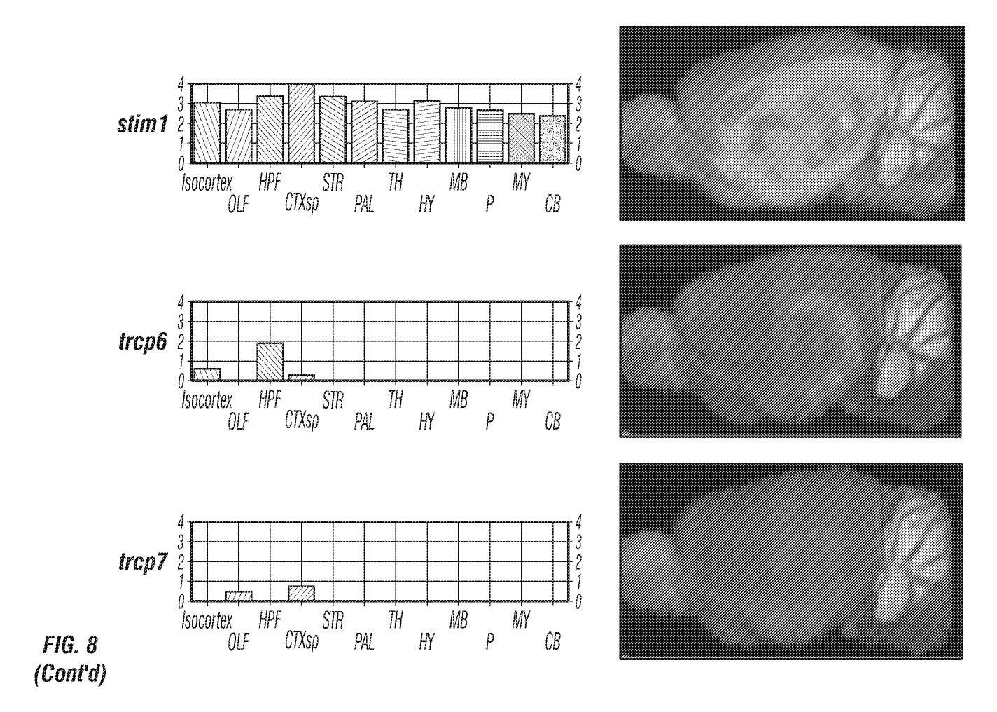

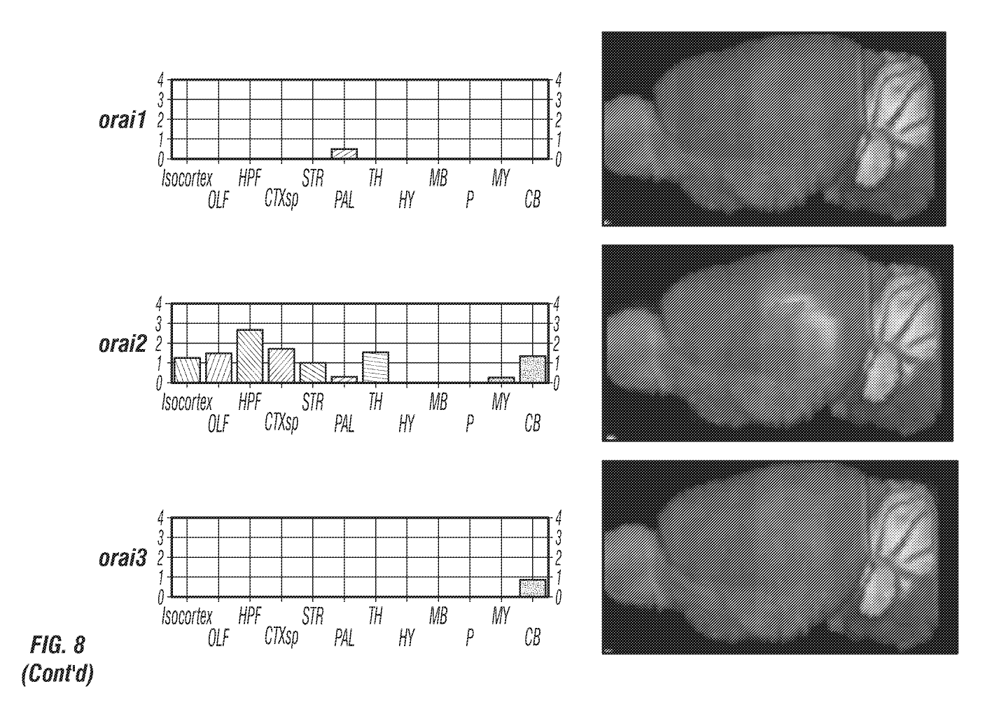

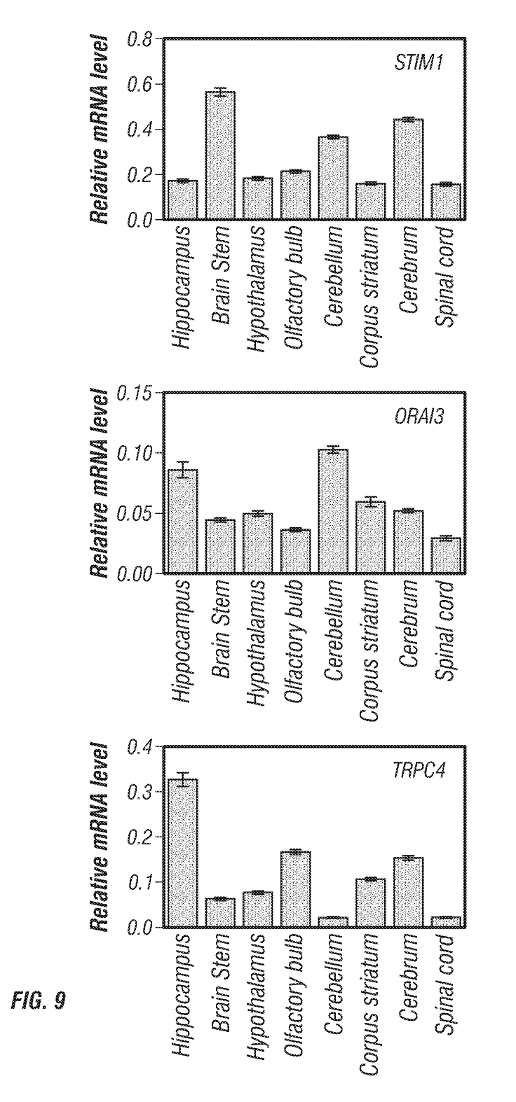

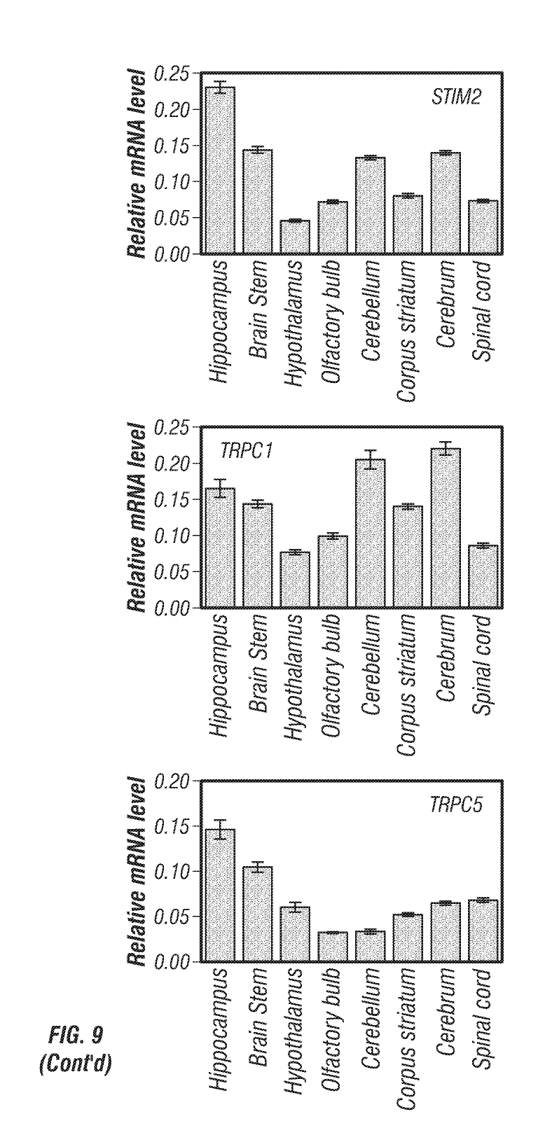

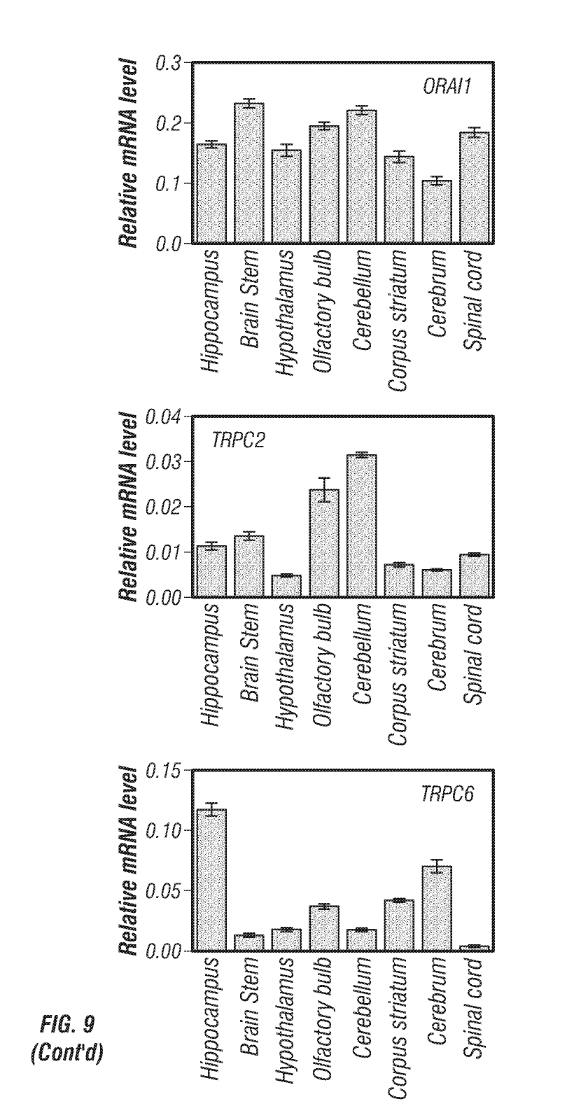

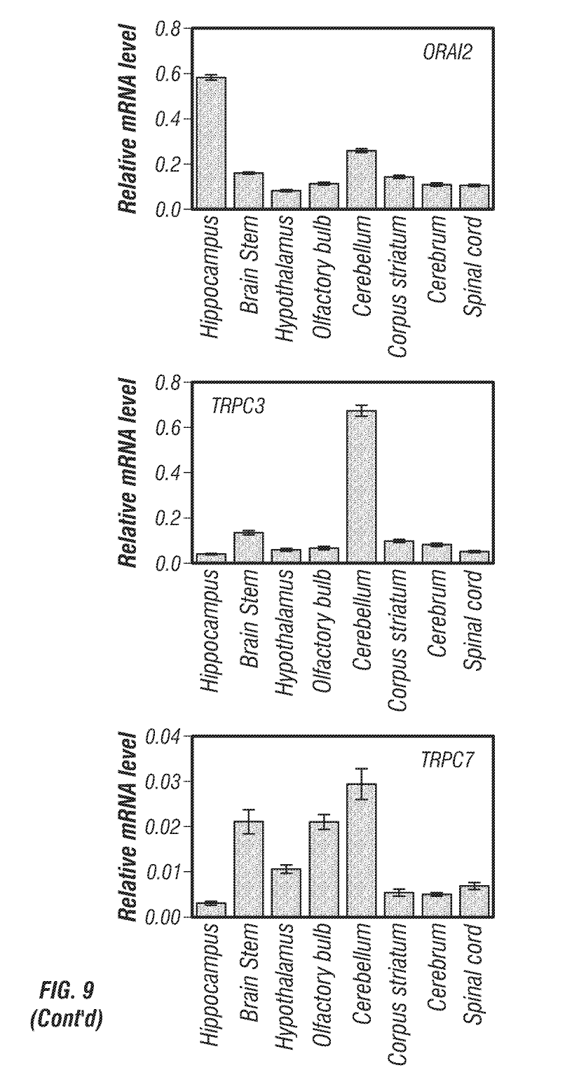

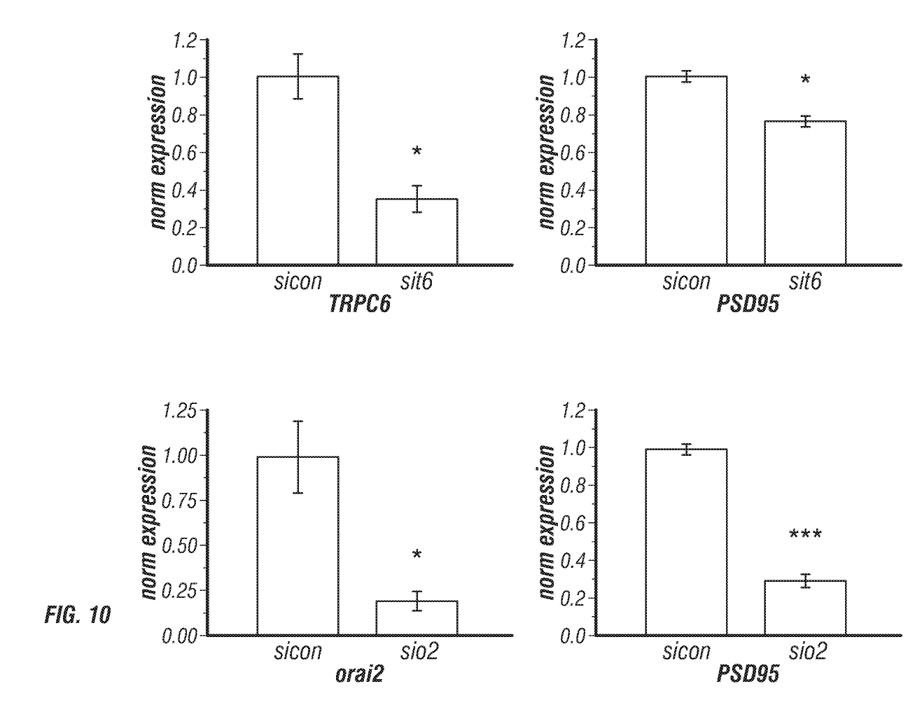

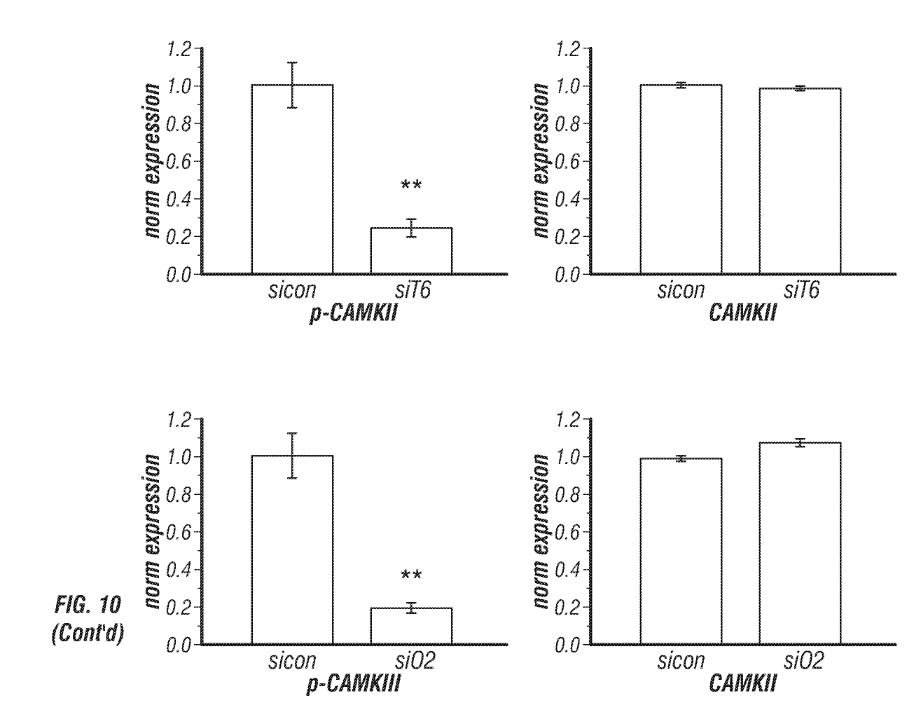

TRPC6 and Orai2 form a complex with STIM2 in the hippocampal synapse. In order to identify molecular components of STIM2-gated nSOC channels in the spines, the inventors took a candidate approach. Previous studies suggested that the two major families of proteins, Transient Receptor Potential Canonical (TRPC) and Orai channels, play a key role in supporting SOC in a variety of cells [Majewski, 2015 #6717; Sun, 2014 #6718]. There are six TRPC proteins in humans (TRPC1, TRPC3TRPC7), which have been divided into two subfamilies, TRPC1/TRPC4/TRPCS and TRPC3/TRPC6/TRPC7, based on biochemical and functional similarities. The remaining member, TRPC2, is a pseudogene in humans but is expressed in other species in a restricted expression pattern [Cheng, 2013 #6719]. There are three Orai channels (Orail-Orai3), but so far most studies have been focused on Orail. The inventors reasoned that the members of TRPC and/or Orai channel families are the most likely candidates to encode STIM2-gated nSOC channels in the spines. Expression of STIM2 is highly enriched in hippocampus [Sun, 2014 #6478] (FIG. 8). The inventors reasoned that other components of STIM2-gated nSOC channels should have a similar expression pattern. Analysis of the data from Allen Brain Atlas revealed that TRPC6 and Orai2 proteins have similar expression pattern (FIG. 8). STIM1 and other members of TRPC and Orai families do not demonstrate significant enrichment in hippocampal region of the brain (FIG. 8). To confirm these results, the inventors performed a series of q-RTPCR experiments with cDNA samples prepared from different brain regions. Consistent with Allen Brain Atlas data, the inventors identified STIM2, TRPC6 and Orai2 as hippocampal-enriched genes (FIG. 9). Based on gene expression data (FIGS. 8-9), the inventors focused on TRPC6 and Orai2 as the candidate molecules encoding STIM2-gated nSOC channel in hippocampal spines.

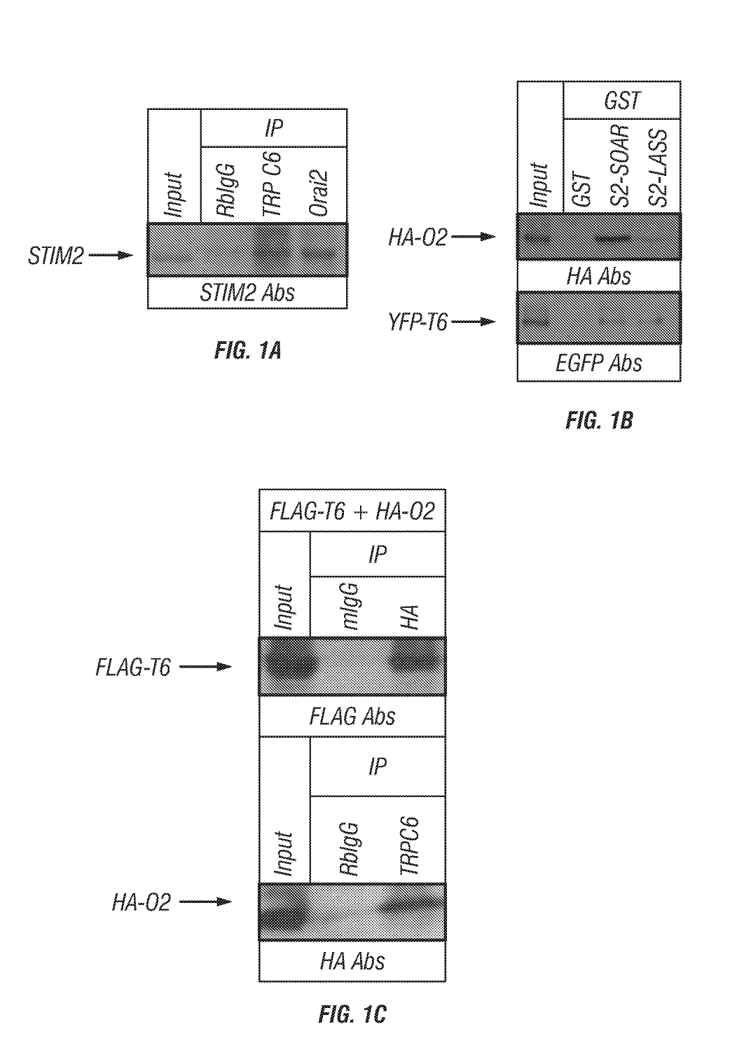

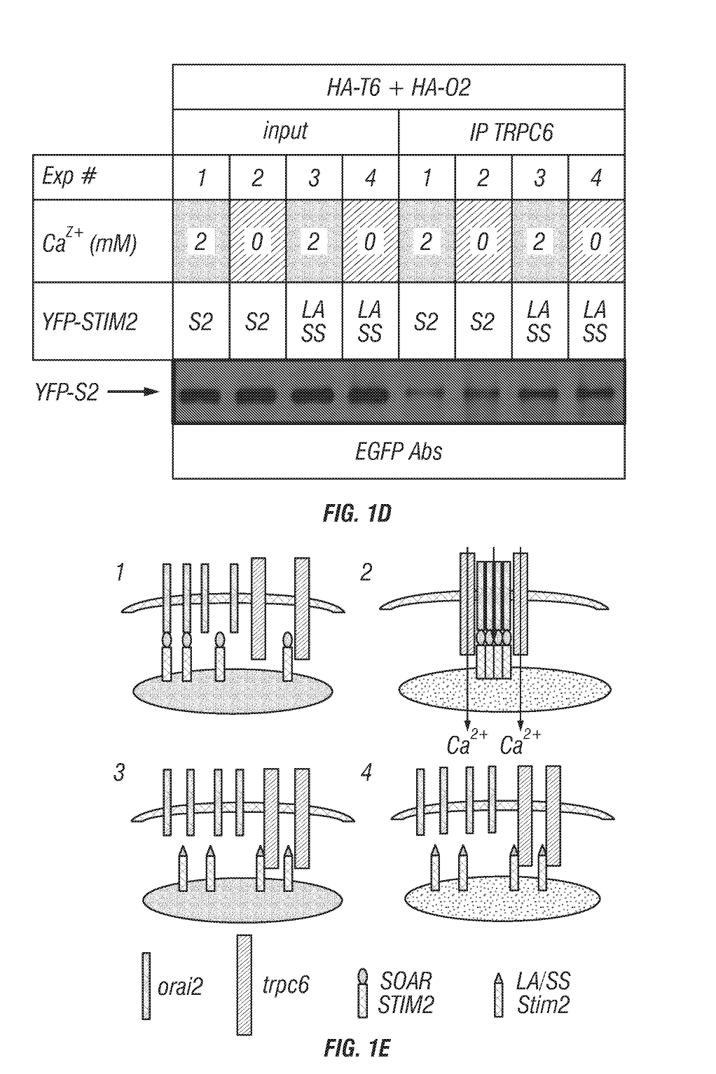

To identify components of STIM2-gated channel complex, the inventors prepared hippocampal synaptosomes and performed a series of immunoprecipitation experiments. They discovered that antibodies against TRPC6 or Orai2 can indeed pull down STIM2 from synaptosomal lysates (FIG. 1A). The apparent molecular weight of immunoprecipited STIM2 was higher than the molecular weight of STIM2 in the input lane (FIG. 1A). It is likely that TRPC6 and Orai2 form a complex with STIM2 that undergone posttranslational modification, such as phosphorylation [Smyth, 2012 #6763]. Consistent with this observation, STIM2 co-immunoprecipitated with Orail from cortical lysates also display higher molecular weight on the gel [Gruszczynska-Biegala, 2013 #6722].

Does STIM2 bind directly to TRPC6 and/or Orai2? STIM1 and STIM2 proteins share similar domain structure and 76% sequence similarity [Stathopulos, 2013 #6729]. STIM2 protein has not been extensively studied, but structure-functional analysis of STIM1 protein has been performed previously by several laboratories. It has been established that STIM1 protein interacts with and gates Orail via a cytosolic SOAR domain [Park, 2009 #6730; Yuan, 2009 #6731]. A double mutation in STIM1 SOAR domain sequence (L373S, A376S) disrupted association between STIM1 and Orai [Frischauf, 2009 #6716]. Guided by sequence homology between STIM1 and STIM2, the inventors generated GST-fusion construct of the wild-type STIM2-SOAR domain (S2-SOAR) and the corresponding mutant (L377S, A380S) in the STIM2-SOAR sequence (S2-LASS). They used these constructs in pull-down experiments with lysates from HEK293 cells transfected with YFP-tagged TRPC6 or HA-tagged Orai2. They discovered that STIM2-SOAR domain strongly associated with Orai2 protein, and that this association was disrupted by LASS mutation (FIG. 1B). In contrast, association of STIM2-SOAR domain with TRPC6 was weak and was not affected by LASS mutation (FIG. 1B). Similar results were observed when the whole cytosolic tail of STIM2 was used in pull-down experiments instead of SOAR domain (data not shown). These results suggested that STIM2 is associated strongly and directly with Orai2 via SOAR domain, but associated with TRPC6 very weakly. This is consistent with previous analysis of STIM1, which has been shown to interact with Orail via SOAR domain and with TRPC1/2/4 but not with TRPC3/6/7 via ERM domain [Huang, 2006 #6732]. To explain the ability of TRPC6 antibodies to precipitate STIM2 (FIG. 1A), the inventors reasoned that Orai2 and TRPC6 may form a complex in the membrane. A similar complex has been proposed previously for TRPC6/3 and Orail in non-excitable cells [Liao, 2007 #6733; Jardin, 2009 #6738]. To test this hypothesis, they co-transfected FLAG-tagged TRPC6 and HA-tagged Orai2 to HEK293 cells and confirmed formation of TRPC6/Orai2 complex in co-immunoprecipitation experiments (FIG. 1C).

To further investigate the function of STIM2-Orai2-TRPC6 complex, the inventors co-transfected HEK293 cells with HA-tagged TRPC6 and Orai2 constructs and YFP-tagged STIM2 construct or YFP-tagged STIM2-LASS mutant construct. Results in the literature suggest that association between STIM1, Orai and TRPC channels can be modulated by depletion state of ER Ca²⁺ stores [Cheng, 2013 #6719; Liao, 2007 #6733; Liao, 2008 #6734; Liao, 2009 #6735; Cheng, 2008 #6736; Cheng, 2011 #6737; Jardin, 2009 #6738; Ong, 2007 #6739; Zeng, 2008 #6740]. To account for this possibility, the inventors prepared lysates from transfected HEK239 cells in standard culture conditions (2 mM extracellular Ca²⁺) following incubation in Ca²+-free media to cause store depletion. The lysates were precipitated with anti-TRPC6 antibodies and presence of YFP-STIM2 was analyzed by Western blotting with anti-EGFP antibodies. In these experiments, the inventors found that under normal Ca²⁺ condition (2 mM Ca²⁺), STIM2 associated with TRPC6 weakly (FIG. 1D, lane 1) and this association was facilitated by store depletion (FIG. 1D, lane 2). Interestingly, LASS mutation in STIM2-SOAR domain sequence did not disrupt association of STIM2 with TRPC6, but this interaction was no longer modulated by ER Ca²⁺ levels (FIG. 1D, lanes 3 and 4). To explain these results, the inventors proposed a model depicted on FIG. 1E. The inventors propose that in conditions of normal ER Ca²⁺ stores, there are some STIM2 proteins that interact strongly with Orai2 via SOAR domain and some STIM2 proteins that interact weakly with TRPC6 via a different region (FIG. 1E, panel 1). Following ER store depletion and oligomerization of STIM2, more Orai2 and TRPC6 proteins are recruited and a functional complex of TRPC6, Orai2 and STIM2 is assembled (FIG. 1E, panel 2). In this complex, TRPC6 serves as a Ca²+-conducting channel and Orai2 is involved in sensing ER Ca²⁺ levels by means of association with STIM2. Similar ideas have been proposed before to explain function of STIM1-TRPC3/6-Orail complex [Liao, 2007 #6733; Jardin, 2009 #6738]. The inventors further argue that LASS mutation in STIM2-SOAR domain disrupts its association with Orai2 and results in enhanced non-productive association with TRPC6 due to loss of competition with Orai2 (FIG. 1E, panel 3). Because of inability to bind Orai2, STIM2-LASS association with TRPC6 is no longer regulated by ER Ca²⁺ store depletion (FIG. 1E, panel 4). The remainder of the discussion will be guided by this model (FIG. 1E) to evaluate function of TRPC6/Orai2-STIM2 complex in hippocampal synaptic spines.

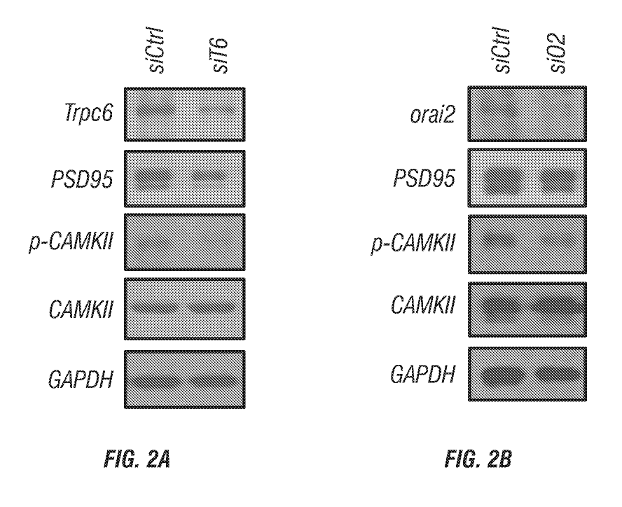

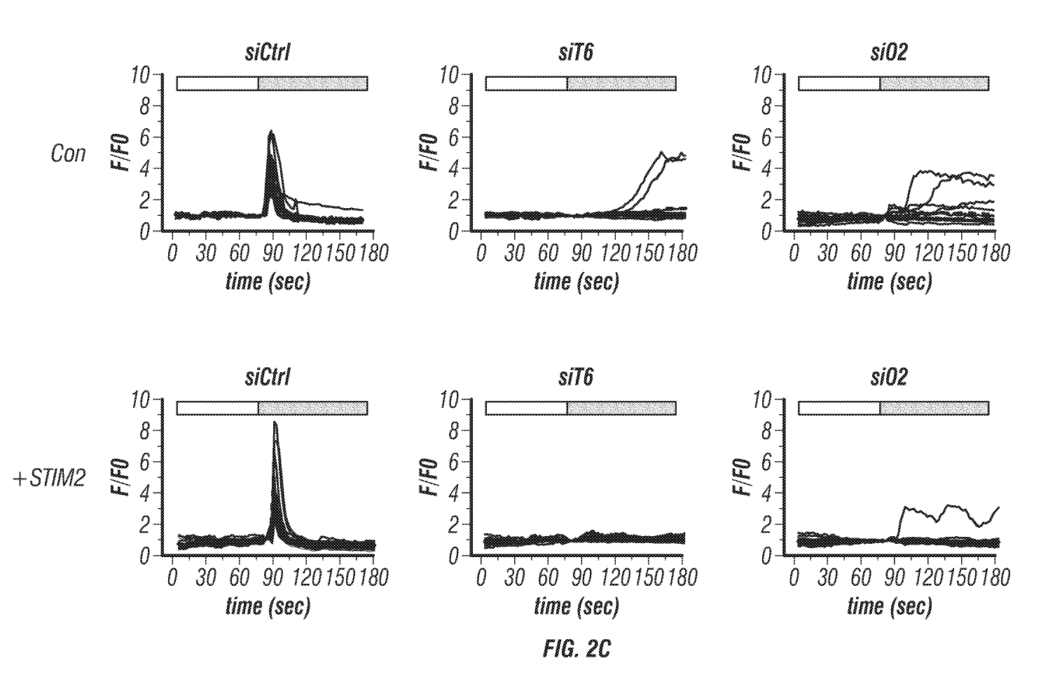

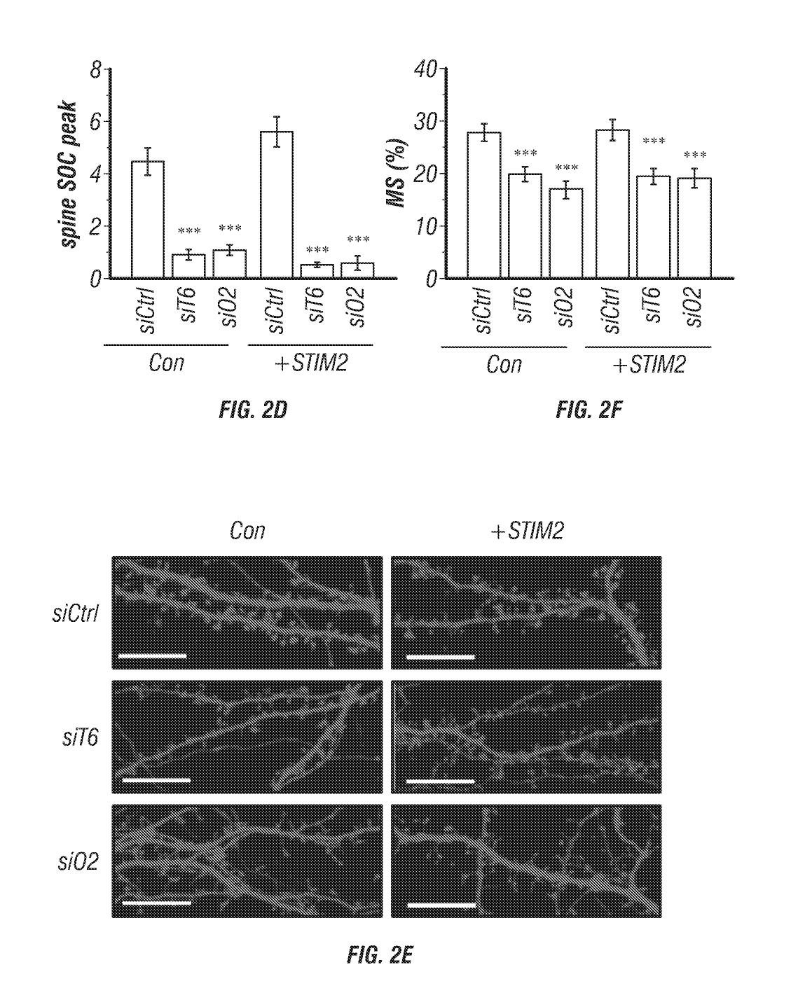

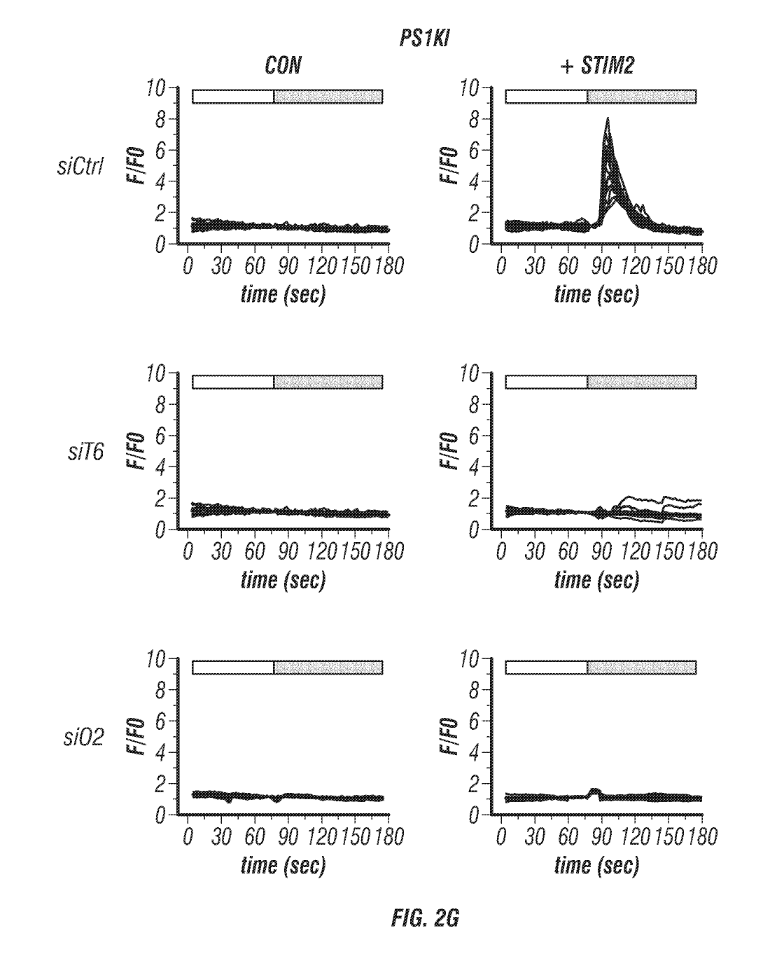

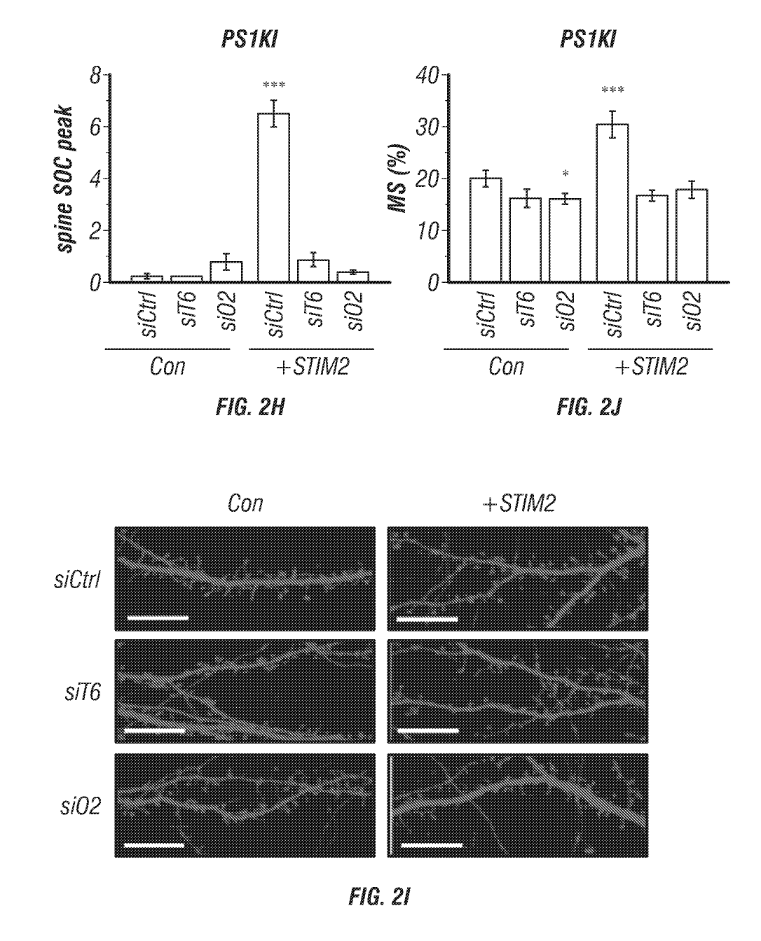

TRPC6 and Orai2 are components of STIM2-gated nSOC channels in hippocampal mushroom spines. To determine if TRPC6 and Orai2 are indeed act as components of STIM2-gated nSOC channels in the spines, the inventors performed knockdown of TRPC6 and Orai2 in mouse hippocampal neuronal cultures by using lentiviral-mediated shRNAi delivery. In the previous studies, the inventors demonstrated that activity of synaptic CaMKII is regulated by nSOC pathway and that the levels of autophosphorylated pCaMKII can be used as biochemical readout for steady-state CaMKII activity in the spines (Sun et al., 2014). The inventors also previously demonstrated that inhibition of nSOC results in loss of PSD95 expression in the spines (Sun et al., 2014). In these experiments, the inventors discovered that RNAi-mediated knockdown of TRPC6 or Orai2 resulted in reduction in PSD95 expression and reduced levels of pCaMKII (FIGS. 2A-B and 10). Total levels of CaMKII remained unaffected (FIGS. 2A-B and 10). The reduction of pCAMKII and PSD95 levels following TRPC6 or Orai2 knockdown is consistent with the changes induced by STIM2 reduction or application of nSOC inhibitors in previous studies (Sun et al., 2014).

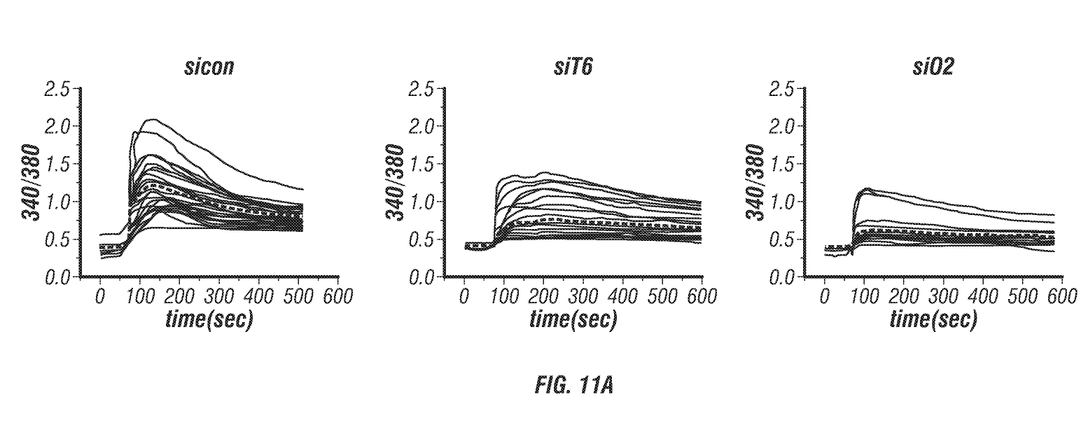

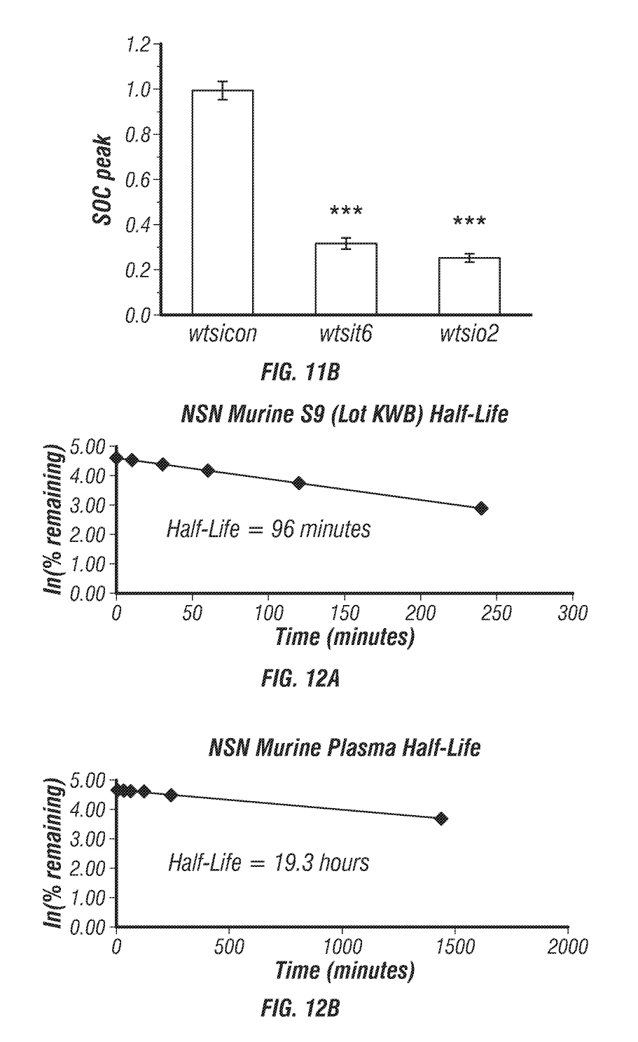

To evaluate nSOC activity more directly, the inventors performed a series of Ca²⁺ imaging experiments. In Fura-2 imaging experiments, the inventors discovered that knockdown of TRPC6 or Oria2 decreased nSOC peak in the soma (FIGS. 11A-B). To perform Ca²⁺ imaging in the spines, the inventors transfected hippocampal neurons with GCamp5.3 plasmid to enable us to simultaneously visualize the dendritic spines and to measure local Ca²⁺ signals (Sun et al., 2014). In these experiments, the inventors discovered that knockdown of TRPC6 or Orai2 resulted in drastic reduction in synaptic nSOC (FIGS. 2C-D). Importantly, overexpression of STIM2 failed to rescue synaptic nSOC following knockdown of TRPC6 or Orai2 (FIGS. 2C-D). These data supported the hypothesis that function of STIM2-gated nSOC channel in the spines requires the presence of both TRPC6 and Orai2 proteins (FIG. 1E, panel 2).

In previous studies, the inventors demonstrated that maintenance of hippocampal mushroom spines requires synaptic nSOC activity (Sun et al., 2014). To evaluate the morphology of synaptic spines following knockdown of TRPC6 and Orai2, the inventors transfected hippocampal cultures with TD-Tomato plasmid, fixed the cells and performed confocal imaging experiments for each experimental group (FIG. 2E). Automated analysis of spine shapes revealed that the fraction of mushroom spines was significantly reduced following knockdown of TRPC6 or Orai2 proteins (FIGS. 2E-F). Similar to nSOC Ca²⁺ imaging experiments, overexpression of STIM2 failed to stabilize mushroom spines following TRPC6 or Orai2 knockdown (FIGS. 2E-F). These data support the hypothesis that STIM2-TRPC6/Orai2 channel complex is necessary for maintenance of hippocampal mushroom synaptic spines.

In a previous publication (Sun et al., 2014), the inventors demonstrated that overexpression of STIM2 can rescue synaptic nSOC and mushroom spine defects in hippocampal neurons from PS1KI mouse model of familial AD. They have been able to replicate these results in the current experiments (FIGS. 2G-J). However, expression of STIM2 failed to rescue synaptic nSOC or mushroom spines in PSI-KT neurons following knockdown of TRPC6 or Orai2 (FIGS. 2G-J). These results further supported the key role of TRPC6 and Orai2 channels in mediating STIM2-gated nSOC in hippocampal mushroom spines.

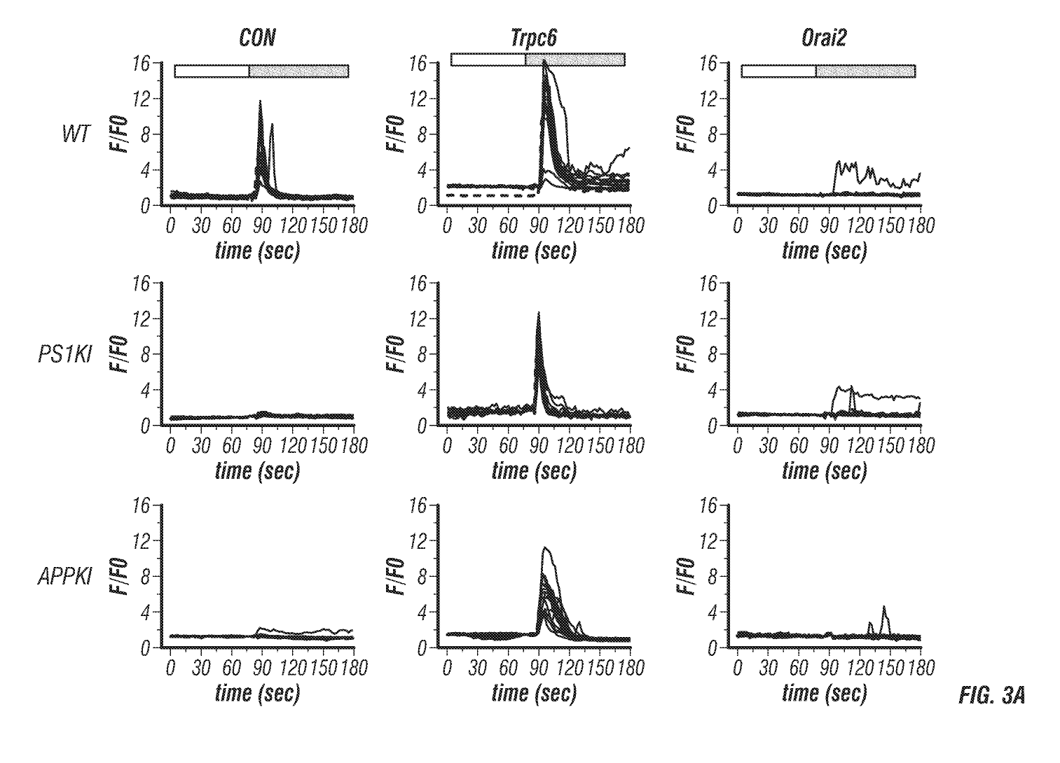

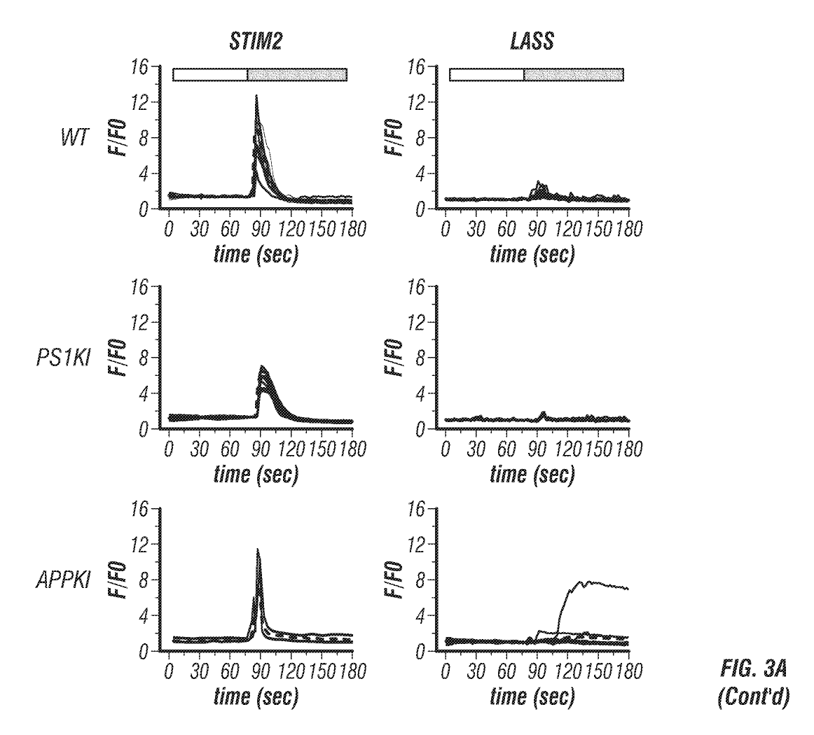

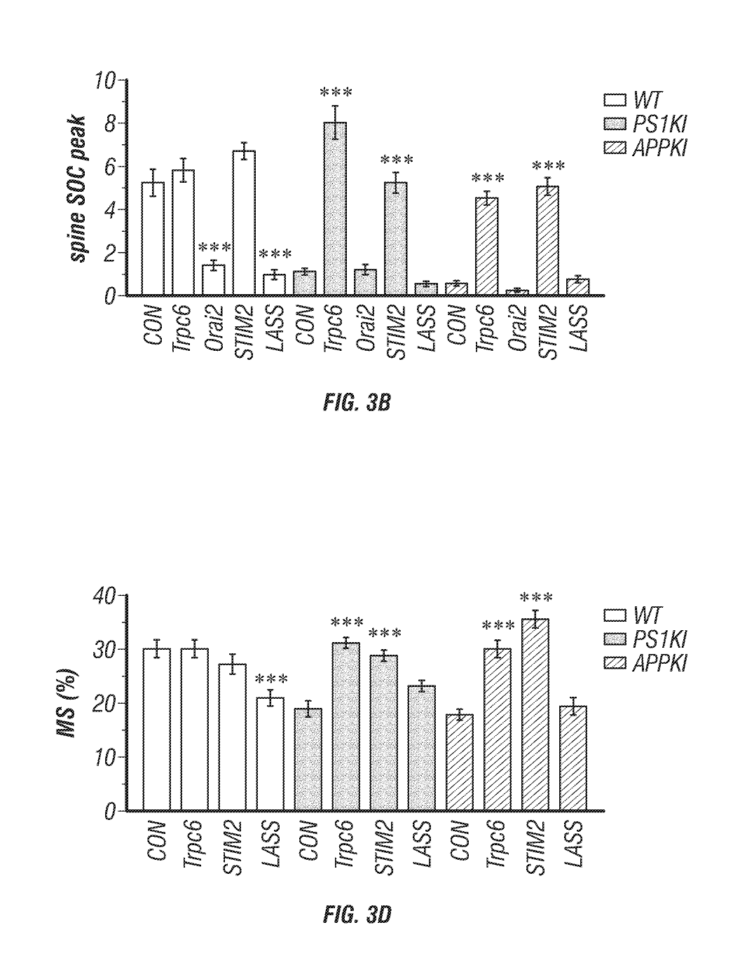

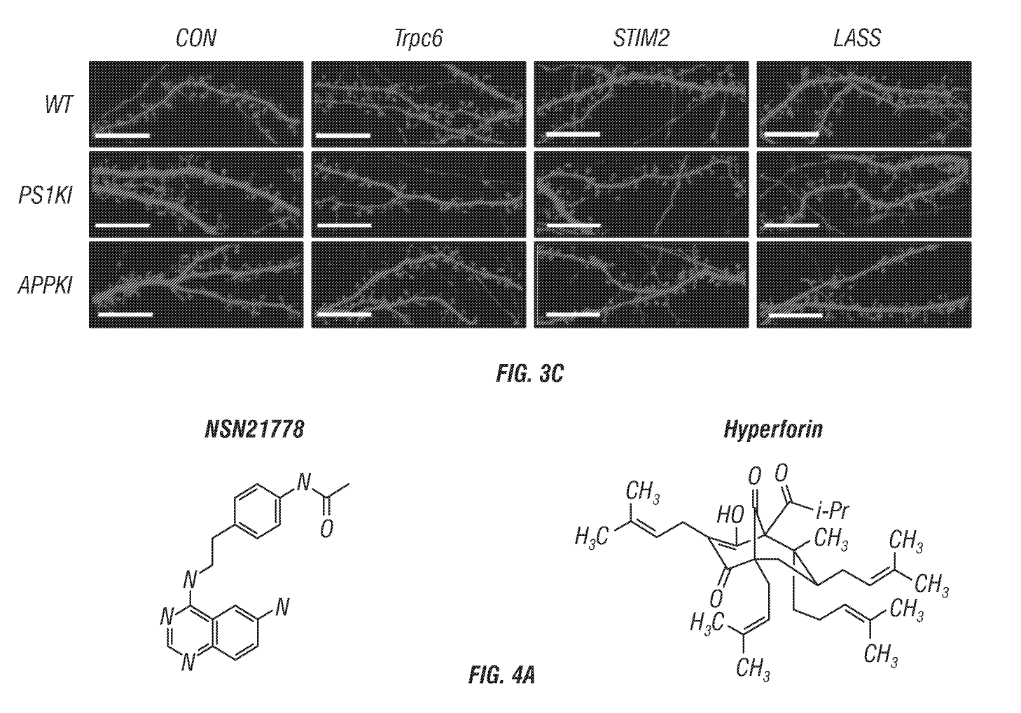

Distinct functional roles of TRPC6 and Orai2 as components of hippocampal spine nSOC. In previous studies, the inventors demonstrated that STIM2 overexpression rescues nSOC and mushroom spine defects in PS1KI and APPKI mouse models of familial AD (Sun et al., 2014 and Zhang et al., 2015). The inventors used similar approach to evaluate effects of TRPC6 and Orai2 overexpression. They determined that overexpression of TRPC6 also rescued spine nSOC (FIGS. 3A-B) and mushroom spines loss (FIGS. 3C-D) in PS1KI and APPKI hippocampal neurons. In contrast, overexpression of Orai2 failed to rescue spine nSOC in PS1KI and APPKI hippocampal neurons (FIGS. 3A-B). In fact, overexpression of Orai2 significantly impaired spine nSOC responses even in wild-type neurons (FIGS. 3A-B). Analysis of spine morphology in Orai2-transfected neurons was not performed, as about 30% of neurons in this group displayed abnormal dendrite morphology and synaptic spines could not be clearly identified in these cells (data not shown). From these results, the inventors concluded that overexpressed Orai2 binds STIM2 with high affinity but does not yield functional Ca²⁺ influx channels in the absence of stochiometric amounts of TRPC6 and/or STIM2. Consistent with these findings, overexpression of Orai1 or Orai2 has been reported to inhibit SOCE by trapping STIM1 in HEK293 cells (Mercer et al., 2006 and Hoover et al., 2011). In contrast, overexpression of TRPC6 results in similar outcomes as overexpression of STIM2, leading to rescue of nSOC and mushroom spines in PS1KI and APPKI hippocampal neurons (FIGS. 3A-D). These results suggest that TRPC6 is the major Ca²⁺ influx source for nSOC pathway. All these results consistent with the model (FIG. 1E, panel 2) that properly regulated nSOC channels in the spines are formed by STIM2-Orai2/TRPC6 complex assembled at correct stoichiometry.

To further test this hypothesis, the inventors compared effects of STIM2 and STIM2-LASS mutant overexpression. In agreement with the previous studies (Sun et al., 2014 and Zhang et al., 2015), expression of STIM2 rescued spine nSOC (FIGS. 3A-B) and mushroom spines loss (FIGS. 3C-D) in PS1KI and APPKI hippocampal neurons. In contrast, expression of STIM2-LASS mutant failed to rescue spine nSOC (FIGS. 3A-B) and mushroom spines loss (FIGS. 3C-D) in PS1KI and APPKI hippocampal neurons. In fact, expression of STIM2-LASS mutant exerted dominant-negative effect on spine nSOC in wild-type neurons (FIGS. 3A-B) and resulted in mushroom spines loss in these neurons (FIGS. 3C-D). To explain these results, the inventors reasoned that STIM2-LASS mutant does not bind to Orai2 and instead traps TRPC6 in non-functional complex (FIG. 1E, panel 4). From these results, the inventors concluded that TRPC6 is a major Ca²⁺ influx channel in the synaptic spines and Orai2 is a regulatory subunit of the complex that is gated by STIM2 in a store depletion-dependent manner. A similar model has been proposed previously for STIM1-TRPC3/6-Orail complex in non-excitable cells (Liao et al., 2007 and Jardin et al., 2009).

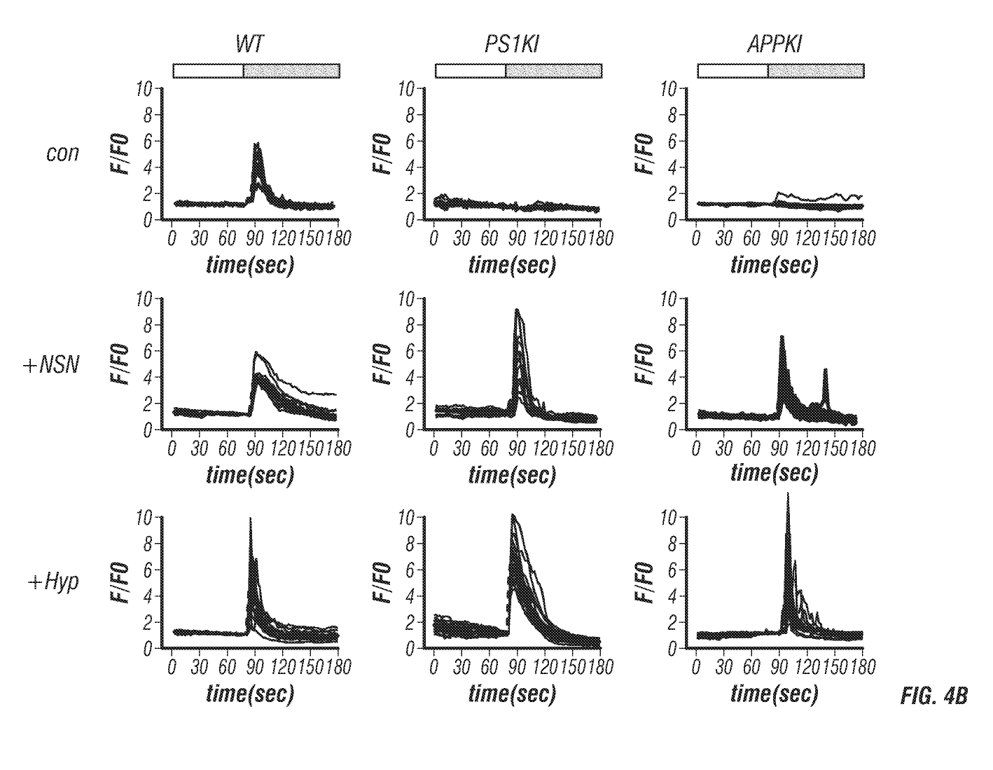

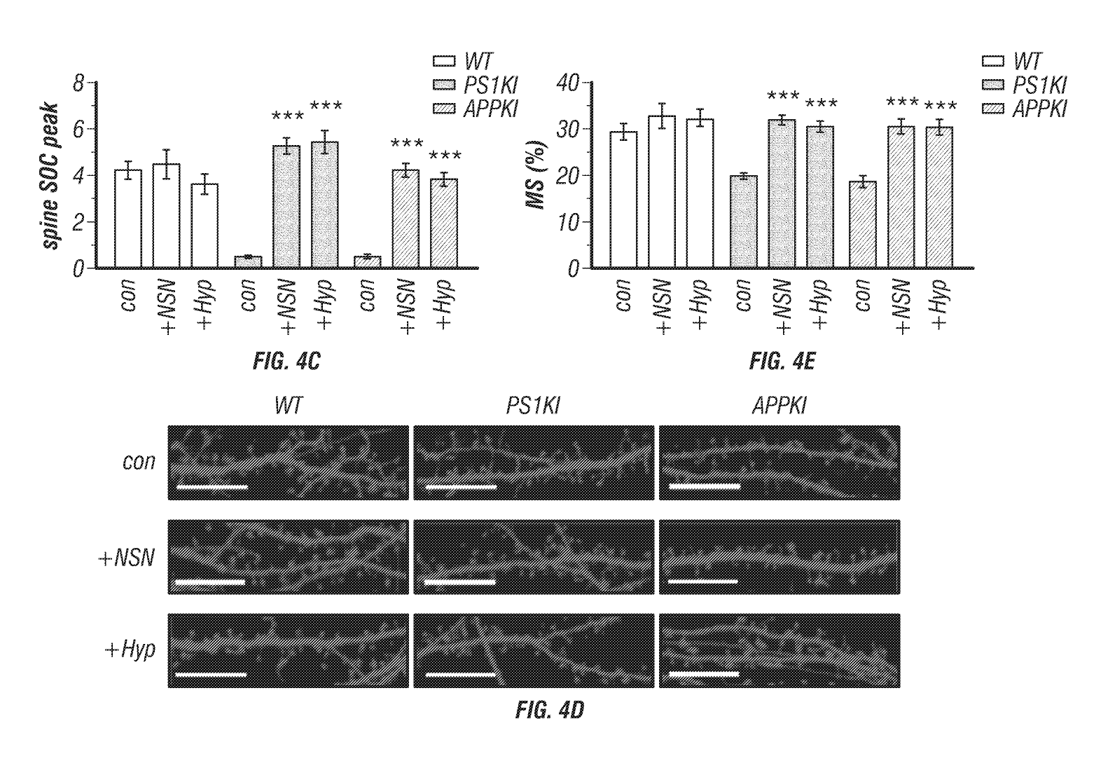

NSN21778 and Hyperforin activate spine nSOC channels. Genetic rescue experiments (Sun et al., 2014 and Zhang et al., 2015) (FIGS. 3A-B) suggest that pharmacological activators of TRCP6/Orai2 nSOC channels in the spines may help to prevent mushroom spine loss and have a therapeutic potential for AD. Hyperforin (Hyp) is a known activator of TRPC6 (Leuner et al., 2007) (FIG. 4A). The inventors' laboratory recently identified a novel nSOC activator NSN21778 (NSN, molecular weight 321) (FIG. 4A). This compound was serendipitously discovered in the process of analyzing novel nSOC inhibitors in a previous study (Wu et al., 2011). In Ca²⁺ imaging experiments, the inventors discovered that application of 300 nM Hyp or NSN prior to Ca²⁺ re-addition rescued spine nSOC in PS1KI and APPKI hippocampal neurons (FIGS. 4B-C). Interestingly, both compounds had no significant effect on nSOC in wild-type spines (FIGS. 4A-B), suggesting that the spine nSOC pathway is already maximally activated in normal conditions. In further experiments, the inventors incubated TD Tomato-transfected hippocampal neuronal cultures with 30 nM concentrations of Hyp or NSN for 16 hr and performed analysis of spine shapes by confocal imaging (FIG. 4D). The inventors discovered that incubation with Hyp or NSN resulted in complete rescue of mushroom spines in both PS1KI and APPKI neurons (FIGS. 4D-E). Both compounds had no significant effect on the fraction of the mushroom spines in wild-type neurons (FIGS. 4D-E). In additional experiments, the inventors demonstrated that 4 hr treatment with 300 nM of Hyp or NSN exerted similar rescue effect on mushroom spines in PS1KI and APPKI hippocampal neurons (data not shown).

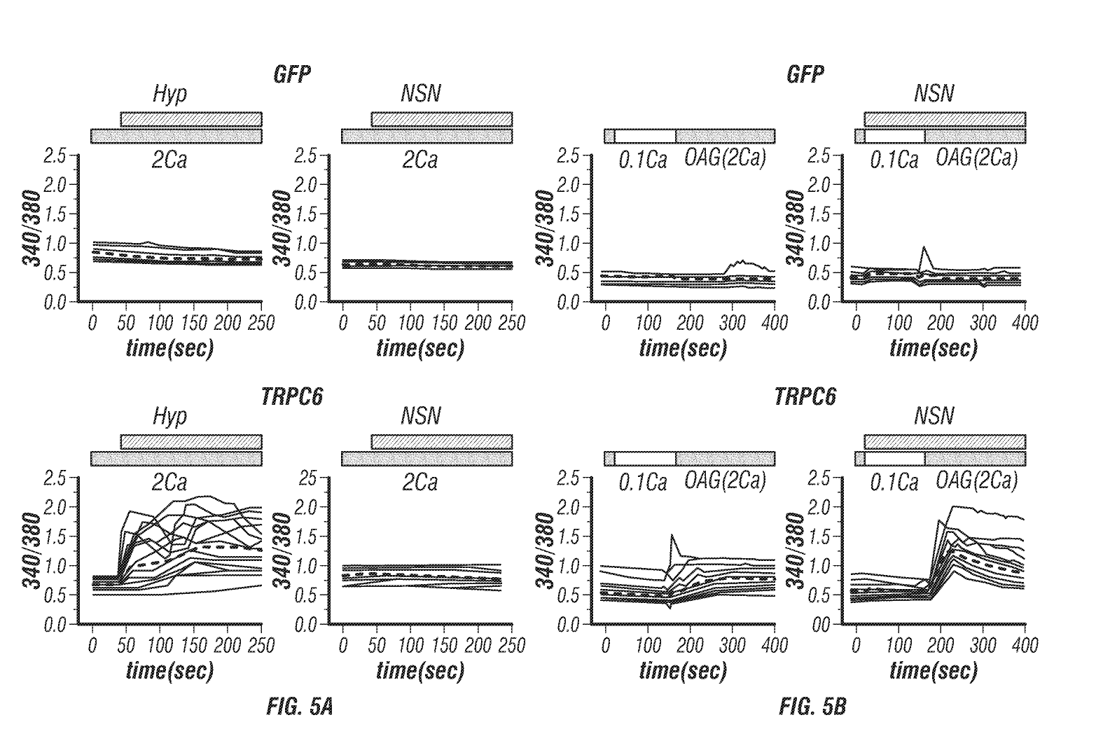

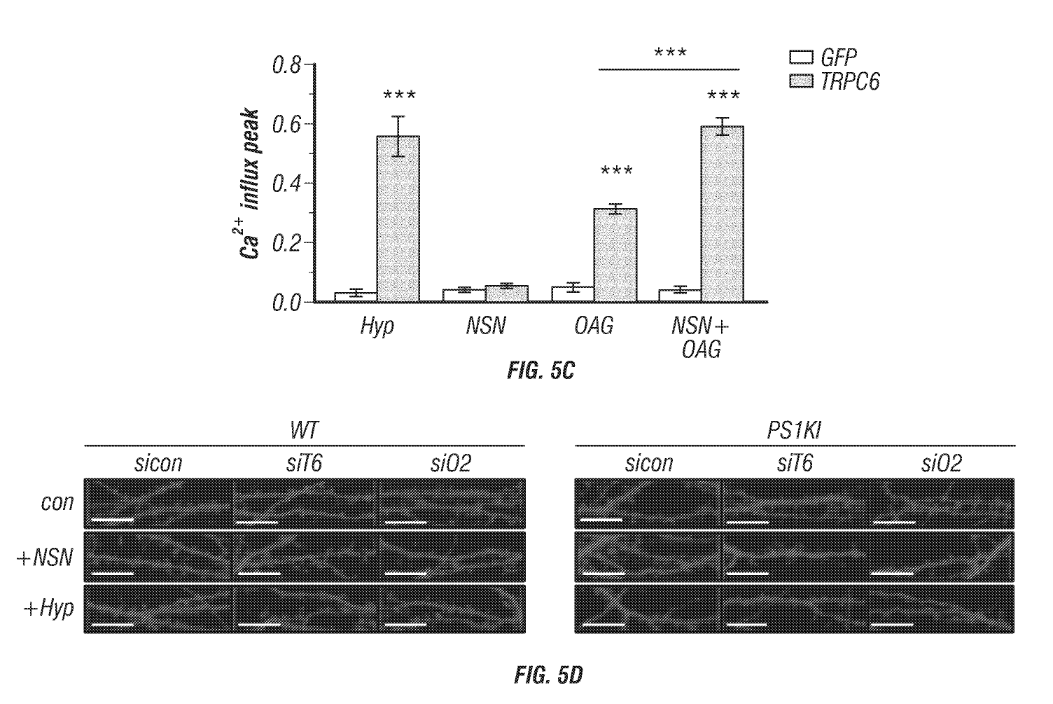

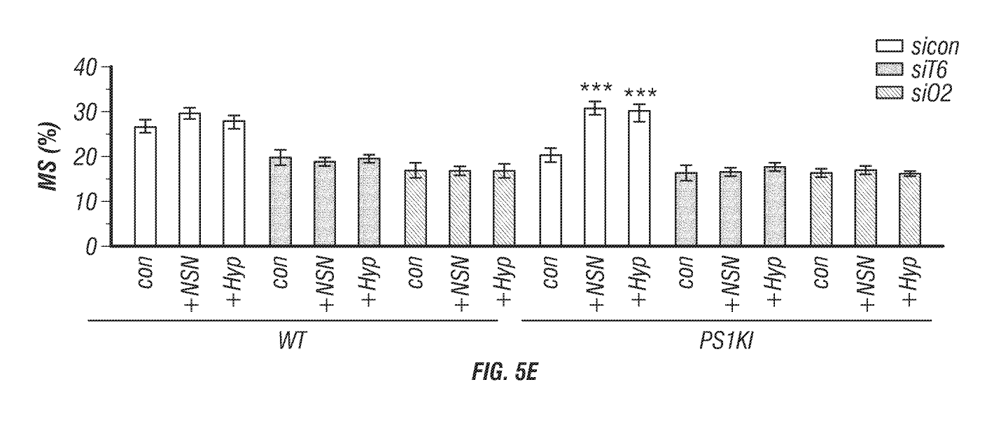

To confirm the target for Hyp and NSN compounds, the inventors overexpressed TRPC6 in HEK293 cells and performed a series of Fura-2 Ca²⁺ imaging experiments. Consistent with the published reports (Leuner et al., 2007), application of 1 μM Hyp activated Ca²⁺ influx in TRPC6-transfected HEK293 cells but not in control cells transfected with EGFP plasmid (FIGS. 5A and 5C). In contrast to Hyp, application of 1 μM NSN did not trigger Ca²⁺ influx in TRPC6-transfected cells (FIGS. 5A and 5C). In additional experiments, the inventors evaluated effects of 1 μM NSN compound in experiments with HEK293 cells transfected with other TRPCs (TRPC1-7), Orais (Orail-3), or combination of TRPC6 and Orai2. However, NSN compound failed to induce Ca²⁺ influx in any of these experiments (data not shown). From these experiments, the inventors concluded that Hyp acts as direct activator of TRPC6 channels, but the actions of NSN compound are more complex. In further experiments, the inventors measured SOC in conditions of store depletion. To achieve this, HEK239 cells were preincubated in Ca²⁺-free media containing 1 μM Tg. In these experiments, the inventors observed endogenous SOC response in EGFP-transfected cells, which was further enhanced in TRPC6-transfected cells. However, application of NSN compound had no additional effect on SOC in GFP or TRPC6 cells in these conditions (data not shown). TRPC6 channels are known to be activated by DAG (Estacion et al., 2004). In spine nSOC measurements, addition of 100 μM DHPG was required to generate robust Ca²⁺ responses. Thus, in the next series of experiments, the inventors evaluated effects of OAG, a synthetic and stable analog of DAG. In standard recording conditions, application of 100 μM OAG to TRPC6-transfected cells resulted in highly variable responses, with some batched of cells displaying strong Ca²⁺ influx and some batches non-responsive (data not shown). However, the inventors discovered that 100 μM OAG can produce more consistent responses when stores are partially depleted by preincubation of cells in extracellular media containing 0.1 mM Ca²⁺ (FIGS. 5B-C). The effect of OAG was observed in TRPC6-transfected cells, but not in control EGFP-transfected cells (FIGS. 5B-C). Interestingly, similar partial store-depletion protocol was used in the paper that reported cloning of STIM2 (Brandman et al., 2007), suggesting that STIM2-dependent Ca²⁺ influx pathways are important in such conditions. Preincubation with 1 μM NSN resulted in significant potentiation of OAG-induced responses in these conditions in TRPC6-transfected cells but not in control GFP cells (FIGS. 5B-C). From these results, the inventors concluded that NSN compound likely to act by potentiating effects of endogenous DAG on TRPC6 channels in neurons.

To validate the target for Hyp and NSN compounds in the spines, the inventors performed experiments with wild-type and PS1KI hippocampal neurons infected with Lenti-RNAi against TRPC6 or Orai2. These cultures were transfected with TD Tomato, incubated with 30 nM of Hyp or NSN for 16 hours and analyzed by confocal microscopy (FIGS. 5A-B). Knockdown of TRPC6 or Orai2 resulted in loss of mushroom spines in wild-type neurons in these experiments (FIGS. 2E-F and 5A-B). Incubation with 30 nM Hyp or NSN failed to rescue this phenotype (FIGS. 5A-B). Incubation with 30 nM Hyp or NSN rescued mushroom spine loss in PS1KI cultures infected with control RNAi lentiviruses, but failed to rescue mushroom spine loss in PS1KI hippocampal neurons following knockdown of TRPC6 or Orai2 (FIGS. 5A-B). The obtained results are consistent with the hypothesis that Hyperforin and NSN rescue synaptic nSOC and mushroom spines in AD neurons by activating TRPC6/Orai2 channel complex in the spines. Effects of Hyperforin and its derivatives in AD mouse models have been previously described (see Discussion), and the inventors focused on the analysis of NSN compound for the remainder of the study.

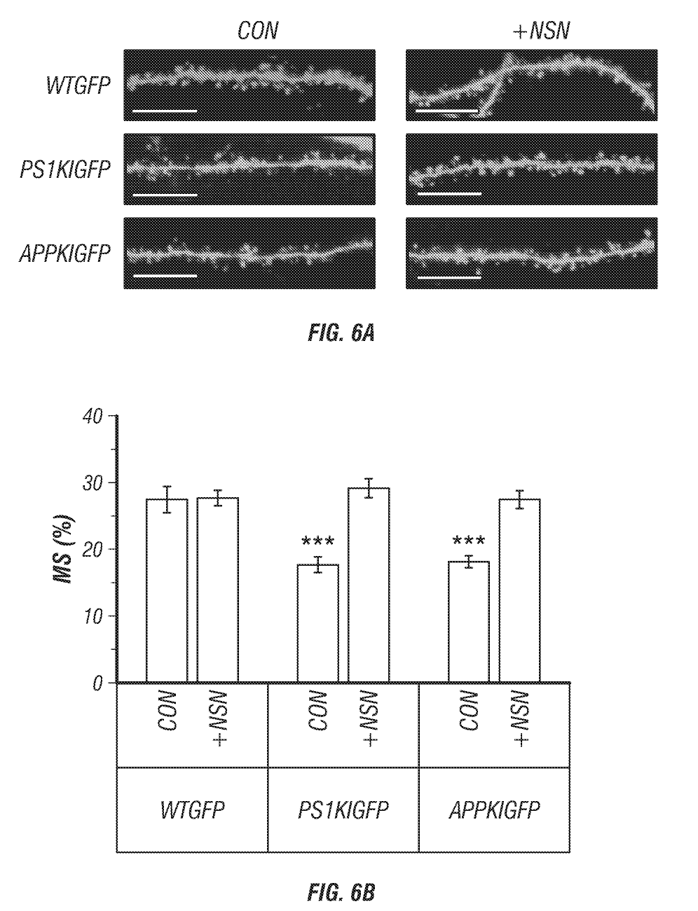

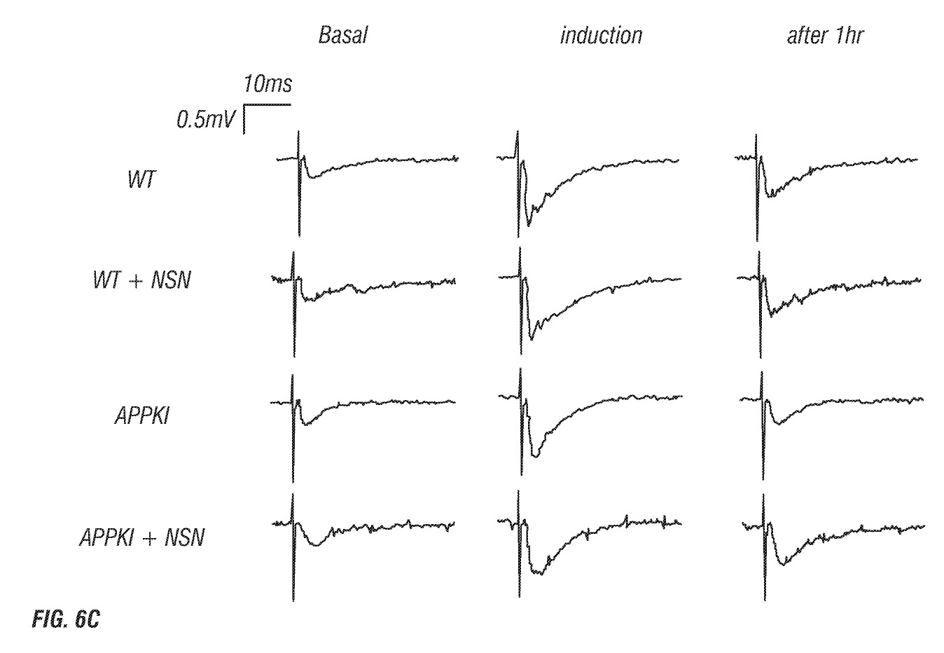

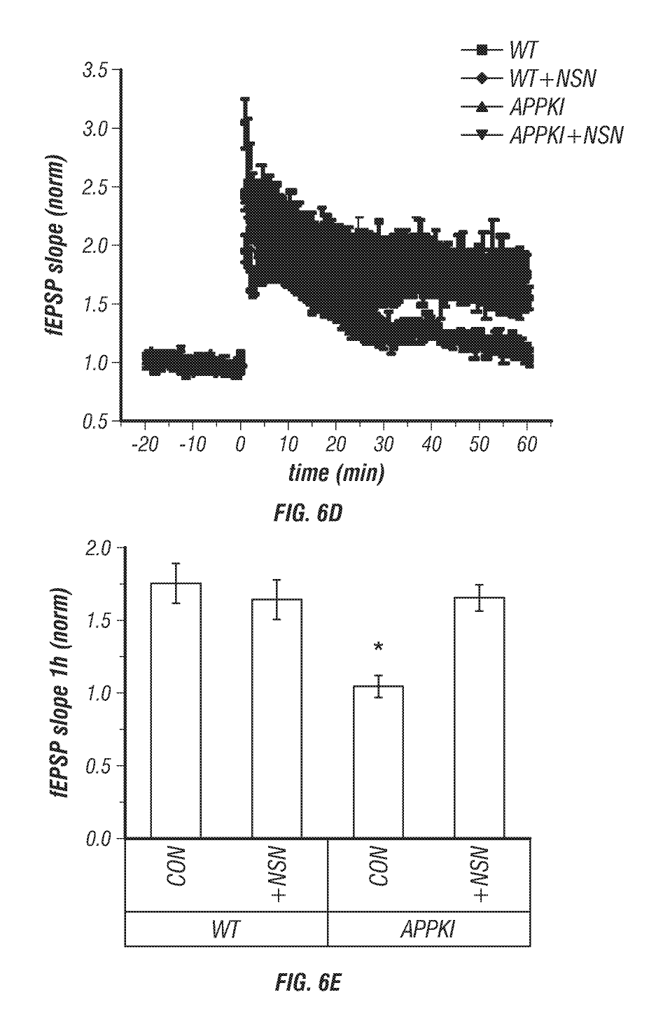

NSN21778 rescues synaptic spine and plasticity defects in hippocampal slices from AD mouse models. To further evaluate synaptic effects of NSN compound, the inventors performed a series of experiments with hippocampal slices. To simplify the analysis, the inventors crossed Line M GFP mice (Feng et al., 2000) with PS1KT and APPKI mice to yield PS1KIGFP and APPKIGFP mice. Hippocampal slices were prepared from 6 month-old Line M GFP mice (WTGFP), PS1KIGFP and APPKIGFP mice. The slices were treated with 300 nM NSN for 3.5 hours, fixed and analyzed by two-photon imaging (FIG. 6A). Consistent with previous studies (Sun et al., 2014 and Zhang et al., 2015), analysis of spine shapes revealed significant loss of mushroom spines in 6 month-old PS1KIGFP and APPKIGFP mice when compared to WTGFP mice (FIGS. 6A-B). Treatment with 300 nM NSN had no effect on mushroom spines in WTGFP mice, but resulted in complete rescue of mushroom spines in PS1KIGFP and APPKIGFP hippocampal slices (FIGS. 6A-B). PS1KI mice do not display E-LTP defects (Chakroborty et al., 2009 and Oddo et al., 2003), and only L-LTP phenotype was reported for these mice (Auffret et al., 2010 and Zhang et al., 2015). APPKI mice have been recently generated (Saito et al., 2014) and no LTP studies have been performed with these mice so far. In these studies, the inventors discovered that two trains of high frequency stimulation (HFS) can induce similar synaptic potentiation in 6 month-old wild-type and APPKI hippocampal slices (FIGS. 6C-D). However, this potentiation did not last in APPKI hippocampal slices (FIGS. 6C-D). On average, for APPKI slices the slope of fEPSP dropped back to pre-stimulation levels within 60 min (FIGS. 6C-6E). In contrast, the slope of fEPSP stayed elevated for wild-type slices, with an average increase of 175% at 60 min time point (FIGS. 6C-6E). These results suggested that APPKI mice display robust LTP defect at 6 months of age, which could be expected from previously described Ar342 effect on hippocampal LTP (Chapman et al., 1999; Shankar et al., 2007; Shankar et al., 2008 and Walsh et al., 2002). Pre-treatment of hippocampal slices with 300 nM NSN compound for 2-3 hours had no significant effect on LTP in wild-type slices but completely rescued LTP defect in APPKI slices (FIGS. 6C-6E). From these experiments, the inventors concluded that activation of spine nSOC pathway by the NSN compound can rescue synaptic plasticity defects in APPKI hippocampal neurons.

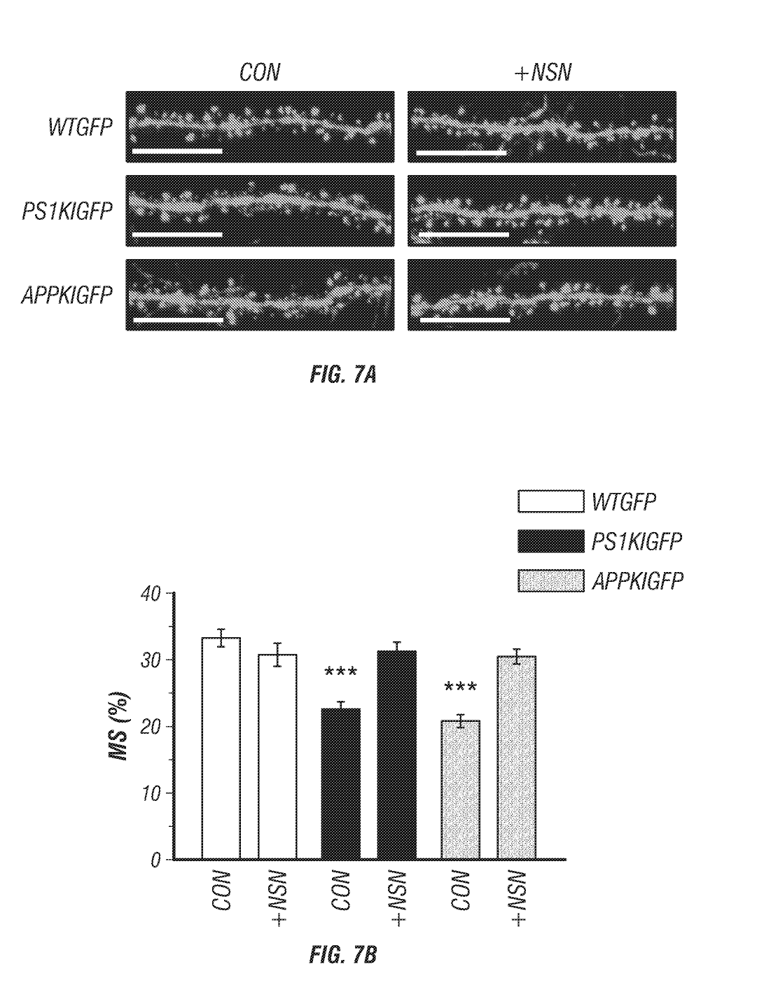

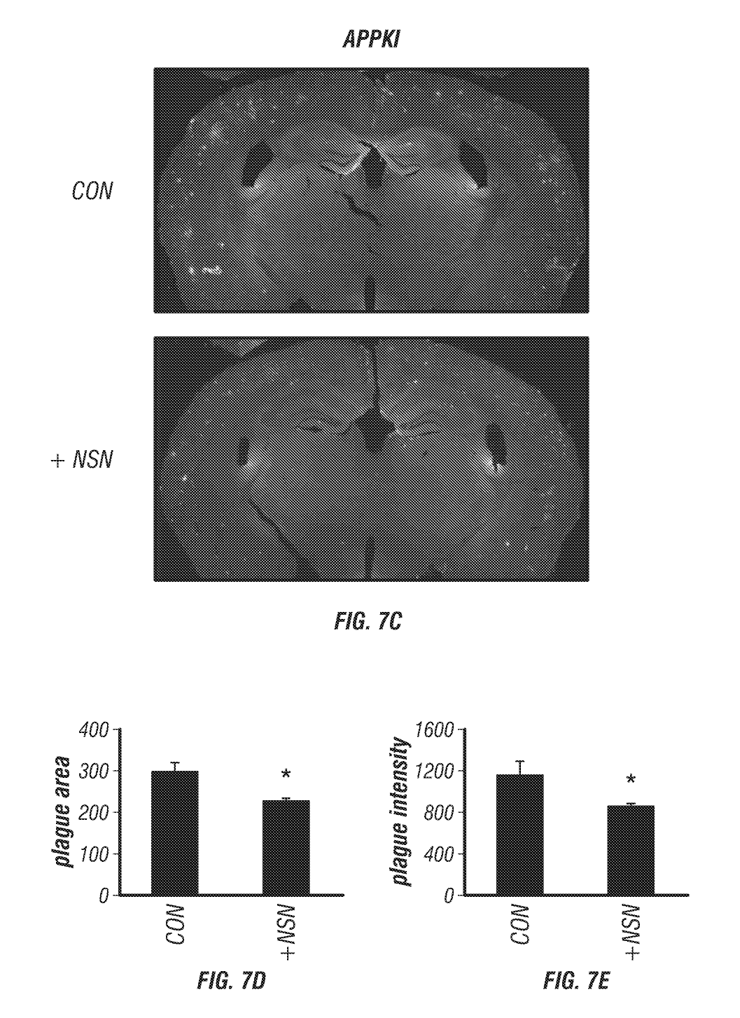

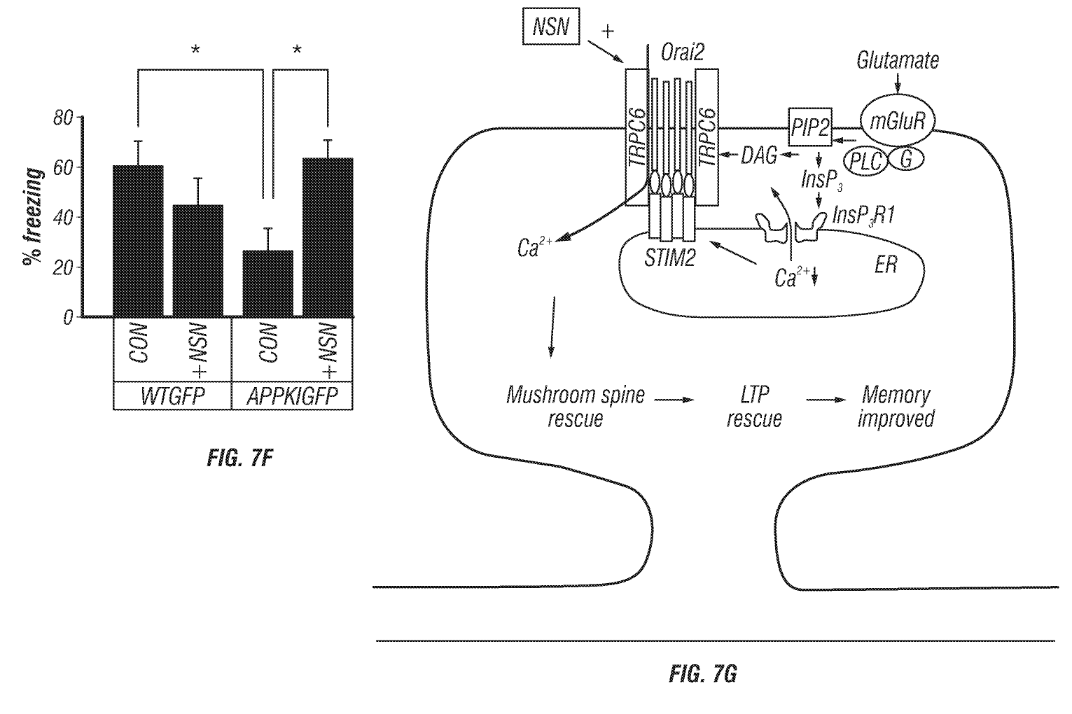

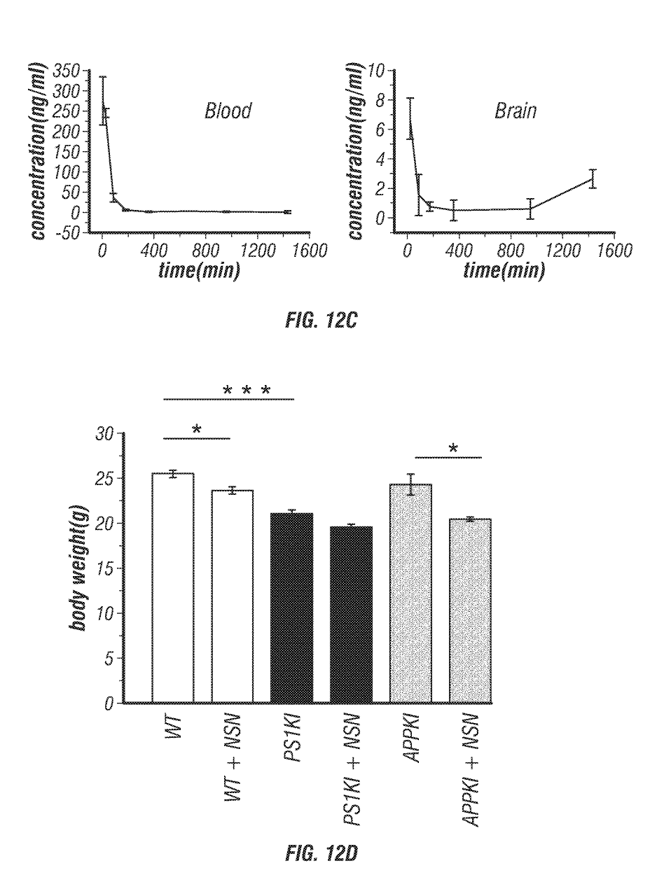

NSN21778 rescues mushroom spines and memory defects in AD mouse models in vivo. To determine if NSN compound can exert beneficial effects in vivo, the inventors performed pilot metabolic stability studies of this compound. They discovered that NSN compound is generally stable in commercial liver S9 fractions in the presence of phase I cofactors, which comprise an NADPH regenerating system, and is stable in commercial CD-1 mouse plasma (FIGS. 12A-B). Following i.p. injection of NSN21778 at 10 mg/kg, the compound reached modest levels in plasma but brain penetration was poor (FIG. 12C). Because NSN compound was effective in nanomolar concentration in spine rescue experiments (FIGS. 4A-D and 5A-B), the inventors initiated whole animal studies nevertheless. In these experiments, NSN compound was injected i.p. 3 times per week at 10 mg/kg concentration in WTGFP, PS1KIGFP and APPKIGFP mice starting from 4 months of age. The inventors have not observed any obvious toxicity in injected mice, but there was some weight loss in NSN-injected mice following 10 weeks treatment (data not shown). The weight loss may result from activation of TRPC6 channels in the gut smooth muscles, which can accelerate intestinal motility (Tsvilovskyy et al., 2009). The mice were sacrificed at 6.5 months of age and analysis of spine shapes was performed by confocal imaging of hippocampal sections (FIG. 7A). Consistent with previous studies (Sun et al., 2014 and Zhang et al., 2015), the inventors observed significant loss of mushroom spines in control group of PS1KIGFP and APPKIGFP mice when compared to WTGFP mice (FIGS. 7A-B). Injections of NSN compound had no effect on mushroom spines in WTGFP mice, but resulted in rescue of mushroom spine deficiency in PS1KIGFP and APPKIGFP mice (FIGS. 7A-B). These results are comparable to in vivo rescue of mushroom spines in PS1KI and APPKI mice following hippocampal injection of AAV1-STIM2 virus (Sun et al., 2014 and Zhang et al., 2015). Thus, the inventors concluded that there is enough NSN penetrating to the brain of these mice following i.p. injections to activate synaptic nSOC and to stabilize mushroom spines. Alternatively, a slightly modified but still active metabolite of NSN, not detected by LC-MS/MS assay, may penetrate the brain in higher concentrations and be responsible for the activity.

Accumulation of amyloid plaques is a hallmark of AD pathology. The APPKI mice start to accumulate amyloid plaques around 12 months of age (Saito et al., 2014). To study the effects of NSN compound on amyloid accumulation, the inventors injected 11 month-old APPKI mice with NSN compound 10 mg/kg. The i.p. injections were performed three times per week for 8 weeks, and the mice were sacrificed at 13 months of age. Consistent with the published observations (Saito et al., 2014), immunostaining revealed accumulation of amyloid plaques in the cortex of APPKI mice (FIG. 7C). The plaques were reduced in the group of mice injected with NSN compound (FIG. 7C). Quantification of the plaque load revealed significant reduction in total plaque area and plaque intensity in NSN-treated mice (FIGS. 7D-E).

The behavioral phenotype of PS1KI mice is very subtle (Wang et al., 2004 and Sun et al., 2005). It has been reported that APPKI mice show only slight impairment in Y-maze assay at 18 months of age (Saito et al., 2014]. However, while handling mice for injection, the inventors noticed after 4 weeks injection, all mice started to show fear memory-related anxious behavior prior to painful injection except the control group of APPKIGFP mice. To formally test memory function in this group of mice, the inventors performed a series of contextual fear conditioning experiments. They indeed discovered that 6.5 month-old APPKIGFP mice have significant impairment in contextual fear conditioning responses when compared to age-matched WTGFP mice (FIG. 7F). The fear conditioning phenotype of APPKIGFP mice was fully rescued by i.p. injections with NSN compound (FIG. 7F). For the WTGFP mice, injections with NSN compound resulted in somewhat impaired responses, but the difference has not reached statistical significance with the control group (FIG. 7F). In addition to contextual fear conditioning, the inventors also performed cued test experiments. A similar behavioral pattern was observed for all 4 groups of mice as in the contextual fear conditioning test, but the difference between the groups did not reach significant level due to large variability in the data within each group (data not shown).

### Example 3

### Discussion

TRPC6 and Orai2 form STIM2-regulated nSOC channel in hippocampal mushroom spines. In previous studies, the inventors demonstrated that STIM2-mediated nSOC in mushroom spines is important for stability of these spines (Sun et al., 2014). They further concluded that nSOC-mediated Ca²⁺ influx causes constitutive activation of synaptic CaMKII, which is necessary for stability of mushroom spines (Sun et al., 2014). Importantly, the inventors demonstrated that STIM2-nSOC-CaMKII pathway is compromised in PS1KI neurons, in APPKI neurons, in aging neurons and in sporadic AD brains due to downregulation of STIM2 protein (Sun et al., 2014 and Zhang et al., 2015). In the present study, the inventors determined the molecular identity of STIM2-gated nSOC channels in hippocampal spines. Starting with the candidate approach, the inventors identified TRPC6 and Orai2 channels as key components of STIM2-gated nSOC. The inventors demonstrated that TRPC6 and Orai2 are enriched in hippocampus (FIGS. 8-9) and biochemically associate with STIM2 and each other (FIGS. 1A-E). Moreover, RNAi-mediated knockdown of TRPC6 or Orai2 suppressed synaptic nSOC and caused loss of mushroom spines in hippocampal neurons (FIGS. 2A-J). These results are consistent with the hypothesis that TRPC6/Orai2 form a functional complex in the mushroom synaptic spines (FIG. 7G). Previously published reports support a potential role of TRPC6 and Orai2 channels in supporting nSOC. Based on neuronal expression pattern it has been previously postulated that Orai2 may play an important role in supporting nSOC (Hoth et al., 2013 and Majewski et al., 2015), but no direct experimental evidence to support this claim has been obtained until these results. TRPC6 have been previously suggested to be critical for spine morphology and neurite growth (Zhou et al., 2008 and Heiser et al., 2013). TRPC6 transgenic mice showed enhancement in spine formation, and spatial learning and memory in Morris water maze (Zhou et al., 2008). Although TRPC6 has been implicated in SOC in some studies, this channel is largely believed to be a receptor-operated channel (ROC) that can be directly activated by diacyl glycerol (DAG) (Sun et al., 2014 and Cheng et al., 2013). In these experiments, the inventors discovered that robust spine nSOC measurements require application of 100 μM DHPG prior to Ca²⁺ add-back. Interestingly, the effects of DHPG were not mimicked by synthetic DAG analog OAG, and direct application of OAG to hippocampal neuronal cultures induced Ca²⁺ responses in just a few spines (data not shown). From these results, the inventors concluded that the activation of TRPC6 channels in the spines requires depletion of the local Ca²⁺ stores and could not be achieved by DAG alone. A similar conclusion is reached based on the experiments with STIM2-LASS mutant. Expression of this mutant, which is not able to interact with Orai2, exerted dominant negative effect on spine nSOC in wild-type neurons (FIGS. 3A-B). During neuronal synaptic activity, activation of mGluR receptors in the spines is coupled to activation of PLC, breakdown of PIP2, generation of DAG and InsP3 and InsP3R1-mediated Ca²⁺ release from ER stores in the spines. These results suggest that activation of TRPC6/Orai2 channels complex in the spines occurs primarily as a result of local ER Ca²⁺ stores depletion and mediated by STIM2 (FIG. 7G). Local generation of DAG may also contribute to activation of TRPC6/Orai2 channels in the spines, but it is not sufficient to activate them without store depletion based on these results. Based on obtained results, the inventors concluded that TRPC6 channel mediates Ca²+ influx in the spines and that Orai2 confers ER Ca²⁺ sensitivity by means of direct interaction with STIM2 (FIG. 7G). Therefore, both TRPC6 and Orai2 are necessary for store depletion-mediated activation of nSOC in synaptic spines. A similar model has been proposed previously for STIM1-TRPC3/6-Orail complex in non-excitable cells (Liao et al., 2007 and Jardin et al., 2009).

TRPC6/Orai2 nSOC channel complex is a novel therapeutic target for AD. These results further indicate that STIM2-gated TRPC6/Orai2 nSOC channel in the spines is a promising therapeutic target for AD and age-related memory loss. In the previous studies, the inventors demonstrated that STIM2 overexpression rescues nSOC and mushroom spine defects in PS1KI and APPKI mouse models of familial AD (Sun et al., 2014 and Zhang et al., 2015). In the present manuscript, the inventors demonstrated that overexpression of TRPC6 also rescued nSOC and mushroom spine defects in PS1KI and APPKI mouse models (FIGS. 3A-B). Moreover, the inventors demonstrated that a known TRPC6 activator Hyperforin and a novel nSOC activator NSN21778 also rescued nSOC and mushroom spine defects in PS1KI and APPKI mouse models (FIGS. 4A-D). It has been demonstrated in the previous studies that hyperforin and its derivatives were able to prevent beta-amyloid neurotoxicity and spatial memory impairments in AβPPSwe/PSEN1ΔE9 (AβPP/PS1) transgenic mice (Inestrosa et al., 2011; Cerpa et al., 2010 and Dinamarca et al., 2006). The mechanism of hyperforin action in these experiments was not clarified. It has been suggested that hyperforin exerts its beneficial effects in these experiments by affecting acetylcholinesterase activity, by reducing AP deposits, by promoting mitochondrial function and neurogenesis (Zolezzi et al., 2013; Carvajal et al., 2013; Abbott et al., 2013; Inestrosa et al., 2011; Cerpa et al., 2010 and Dinamarca et al., 2006). However, in the recent study, it has been suggested that hyperforin derivative tetrahydrohyperforin (IDN5706) rescued Aβ-induced synaptic plasticity defects by activating TRPC3/6/7 channels in neurons (Montecinos-Oliva et al., 2014). It was also recently reported that hyperforin can modulates dendritic spine morphology in hippocampal slice cultures through activation of TRPC6 channels (Leuner et al., 2013). The inventors' results with hyperforin (FIGS. 4A-D) are consistent with the conclusion that hyperforin and its derivatives exert beneficial effects in AD models by stimulation TRPC6-mediated nSOC in mushroom synaptic spines.

The inventors established that both Hyperforin and NSN compound act on Trpc6/Orai2 channel complex, as knockdown of either TRPC6 or Orai2 made these compounds ineffective in spine rescue assay (FIGS. 5A-E). However, the mechanism of action for these two compounds differs. Hyperforin was able to directly activate TRPC6 channels expressed in HEK293 cells in standard recording conditions (FIGS. 5A-E). In contrast, the NSN compound was not effective in these experiments, but it was able to facilitate OAG-induced Ca²⁺ influx through TRPC6 channels in conditions of partially depleted intracellular stores (FIGS. 5A-E). These results suggested that hyperforin acts as direct activator of TRPC6 but NSN compound acts as a positive modulator of TRPC6 (FIGS. 5A-E and FIG. 7G). The exact mechanism of action for NSN compound will require further investigation, but ability of this compound to act as a positive modulator of endogenous spine nSOC channels in physiological conditions may offer additional benefits for therapeutic applications in AD. Indeed, in primary hippocampal culture experiments, the inventors observed very large Ca² elevations and toxicity following treatment with 10 μM hyperforin, suggesting excessive activation of nSOC pathway. They also observed neuronal toxicity in some batches of hippocampal cultures treated overnight with 300 nM of hyperforin (data not shown). In contrast, the inventors did not observe any toxicity in acute experiments with 10 μM of NSN compound or following overnight incubation with 300 nM of NSN compound. These results suggest that NSN compound may have a wider therapeutic window than direct activators of TRPC6 such as hyperforin and its derivatives. In their experiments, the inventors demonstrated that NSN compound was able to rescue mushroom spine loss in hippocampal cultures and slices from PS1KI and APPKI mouse models (FIGS. 4D-E and 6A-B) and rescue hippocampal LTP defects in APPKI mice (FIGS. 6C-E). Moreover, NSN compound rescued mushroom spine loss in PS1KI and APPKI mice when delivered by i.p. injections (FIGS. 7A-B). NSN compound also reduced amyloid load in aging APPKI mice when delivered by i.p. injections (FIGS. 7C-E). It has been reported that nSOC acts as negative regulator of AP production (Zeiger et al., 2013), which may explain the ability of NSN compound to reduce amyloid load in APPKI mice. Importantly, the inventors demonstrated that i.p. injections of NSN compound can rescue APPKI mice memory defects in contextual fear conditioning test (FIG. 7F). Based on obtained results (FIGS. 4A-7G), the inventors concluded that NSN21778 is a promising candidate molecule for therapeutic intervention in brain aging and AD.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. References

The following references, to the extent that they provide exemplary procedural or other details supplementary to those set forth herein, are specifically incorporated herein by reference.

Abbott et al., J Alzheimers Dis 34, 873-885, 2013.

Auffret et al., J Alzheimers Dis 19, 1021-1033, 2010.

Bezprozvanny, I., and Hiesinger, P. R. Mol Neurodegener 8, 23, 2013.

Bourne, J., and Harris, K. M. Do thin spines learn to be mushroom spines that remember? Current opinion in neurobiology 17, 381-386, 2007.

Bourne, J. N., and Harris, K. M. Annu Rev Neurosci 31, 47-67, 2008.

Carvajal et al., J Alzheimers Dis 36, 99-118, 2013.

Cerpa et al., Curr Alzheimer Res 7, 126-133, 2010.

Chakroborty et al., The Journal of Neuroscience 29, 9458-9470, 2009.

Chapman et al., Nature neuroscience 2, 271-276, 1999.

Chartier-Harlin et al. Nature 353:844-846, 1991.

Cheng et al., Curr Top Membr 71, 149-179, 2013.

Cheng et al., PLoS Biol 9, e1001025, 2011.

Cheng et al., The Journal of biological chemistry 283, 12935-12940, 2008.

Citron et al., Nature 360:622-674, 1992.

Corder, Science 261:921-923, 1993.

Dinamarca et al., Mol Psychiatry 11, 1032-1048, 2006.

Estacion et al., Journal of Biological Chemistry, 279:22047-22056, 2004.

Feng et al., Neuron 28, 41-51, 2000.

Feng et al., Neuron 28, 41-51, 2000.

Frischauf et al., The Journal of biological chemistry 284, 21696-21706, 2009.

Glenner and Wong, Biochem. Biophys. Res. Comm. 120:885-890, 1984.

Goate et al. Nature 349:704-706, 1991.

Goldgaber et al., Science 235:877-880, 1987.

Gruszczynska-Biegala et al., Journal of neurochemistry 126, 727-738, 2013.

Guo et al., Nat Med 5, 101-106, 1999.

Heiser et al., Journal of neurochemistry 127, 303-313, 2013.

Hoover, P. J., and Lewis, R. S. Proceedings of the National Academy of Sciences of the United States of America 108, 13299-13304, 2011.

Hoth, M., and Niemeyer, B. A. Curr Top Membr 71, 237-271, 2013.

Huang et al., G. Nature cell biology 8, 1003-1010, 2006.

Inestrosa et al., Transl Psychiatry 1, e20, 2011.

Jardin et al., The Biochemical journal 420, 267-276, 2009.

Kang et al., Nature 325:733-735, 1987.

Kasai et al., Trends Neurosci 26, 360-368, 2003.

Kelly, Microbiol. Sci. 1(9):214-219, 1984.

Koffie et al., Mol Neurodegener 6, 63, 2011.

Kwon, Y., The Handbook of Essential Pharmacokinetics, Pharmacodynamics, and Drug Metabolism for Industrial Scientists (Kluwer Academic/Plenum Publishers), 2001.

Lemaire et al., Nucl. Acids Res. 17:517-522, 1989.

Leuner et al., FASEB journal 21, 4101-4111, 2007.

Leuner et al., Hippocampus 23, 40-52, 2013.

Levy et al., Science 248:1124-1128, 1990.

Levy-Lahad et al., Science 269:973-977, 1995.

Liao et al., Proceedings of the National Academy of Sciences of the United States of America 105, 2895-2900, 2008.

Liao et al., Proceedings of the National Academy of Sciences of the United States of America 104, 4682-4687, 2007.

Liao et al., Proceedings of the National Academy of Sciences of the United States of America 106, 3202-3206, 2009.

Majewski, L., and Kuznicki, J. Biochim Biophys Acta, 2015.

Mann et al., Neurobiol. Aging 10:397-399, 1989.

McNaney et al., Assay Drug Devel Technol 6, 121-129, 2008.

Mercer et al., The Journal of biological chemistry 281, 24979-24990, 2006.

Montecinos-Oliva et al., Current medicinal chemistry 21, 3494-3506, 2014.

Mullan et al., Nature Genet. 1:345-347, 1992.

Murrell et al. Science 254:97-99, 1991.

Oddo et al., Neuron 39, 409-421, 2003.

Ong et al., The Journal of biological chemistry 282, 9105-9116, 2007.

Park et al., Cell 136, 876-890, 2009.

Podlisny et al., Neurobiol. Dis. 3:325-37, 1997.

Popugaeva et al., Messenger 1, 53-62, 2012.

Popugaeva et al., STIM2 protects mushroom synaptic spines from amyloid synaptotoxicity, submitted, 2015.

Popugaeva, E., and Bezprozvanny, I. Front Mol Neurosci 6, 29, 2013. Robakis et al., Proc. Natl. Acad. Sci. 84:4190-4194, 1987.

Rodriguez et al., PLoS One 3, e1997, 2008.

Rodriguez et al., PloS one 3, e1997, 2008.

Rogaev et al., Nature 376:775-778, 1995.

Rumble et al., New England J. Med. 320:1446-1452, 1989.

Saito et al., Nature neuroscience 17, 661-663, 2014.

Selkoe and Schenk, Annu. Rev. Pharmacol. Toxicol. 43:545-584, 2003.

Selkoe, D. J. Nature 399:23-31, 1999.

Selkoe, D. J. Science 298, 789-791, 2002.

Shankar et al., Nat Med 14, 837-842, 2008.

Shankar et al., The Journal of neuroscience 27, 2866-2875, 2007.

Sherrington et al., Nature 375:754-760, 1995.

Sinha & Lieberburg, Proc. Natl. Acad. Sci., USA, 96:11049-11053, 1999.

Smyth et al., Current biology : C B 22, 1487-1493, 2012.

Stathopulos, P. B., and Ikura, M. Curr Top Membr 71, 59-93, 2013.

Sun et al., Cells 3, 455-475, 2014b.

Sun et al., Neurodegener Dis 2, 6-15, 2005.

Sun et al., Neuron 82, 79-93, 2014a.

Suzuki et al., Science 264:1336-1340, 1994.

Tackenberg et al., Curr Alzheimer Res 6, 261-268, 2009.

Tanzi et al., Nature 331:528-530, 1988.

Thinakaran et al., Neuron 17:181-190, 1996.

Tsvilovskyy et al., Gastroenterology 137, 1415-1424, 2009.

Van Broeckhaven et al., Science 248:1120-1122, 1990.

Walsh et al., Nature 416, 535-539, 2002.

Wang et al., Neuroscience 126, 305-312, 2004.

Wolfe et al., Nature 398:513-517, 1999.

Wu et al., Chem Biol 18, 777-793, 2011.

Yoshikai et al., Gene 87, 257-263, 1990.

Yuan et al., Nature cell biology 11, 337-343, 2009.

Zeiger et al., The Journal of biological chemistry 288, 26955-26966, 2013.

Zeng et al., Molecular cell 32, 439-448, 2008.

Zhang et al., J Alzheimers Dis 45, 561-580, 2015a.

Zhang et al., J Alzheimers Dis 45, 561-580, 2015a.

Zhang et al., J Neurosci 25, 1037-1049, 2005.

Zhang et al., J Neurosci 30, 8566-8580, 2010.

Zhang et al., Neuronal store-operated calcium entry and synaptic loss in APP knock-in mouse model of Alzheimer's disease, submitted, 2015b.

Zhang et al., Neuronal store-operated calcium entry and synaptic loss in APP knock-in mouse model of Alzheimer's disease, submitted, 2015b.

Zhang et al., The Journal of neuroscience 30, 8566-8580, 2010.

Zhou et al., Nature neuroscience 11, 741-743, 2008.

Zolezzi et al., J Alzheimers Dis 37, 735-746, 2013.

1. A method of treating a mammalian subject with Alzheimer's Disease comprising administering to said subject a compound wherein the compound is further defined by the formula: 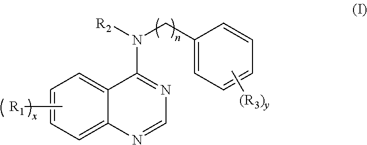
wherein:
each R₁ is independently selected from amino, cyano, carboxyl, halo, hydroxy, or nitro; or
alkylamino_{(C≤8)}, dialkylamino_{(C≤8)}, cycloalkylamino_{(C≤8)}, dicycloalkylamino_{(C≤8)}, or a substituted version of any of these groups;
x is 1, 2, 3, 4, or 5;
R₂ is hydrogen, alkyl_{(C≤8)}, or substituted alkyl_{(C≤8)};
n is 1, 2, 3, 4, or 5;
each R₃ is independently selected from amino, carboxyl, cyano, halo, hydroxy, or nitro; or
alkyl_{(C≤8)}, cycloalkyl_{(C≤8)}, alkenyl_{(C≤8)}, alkynyl_{(C≤8)}, acyl_{(C≤8)}, amido_{(C≤8)}, or a substituted version of any of these groups; and
y is 1, 2, 3, 4, or 5;
or a pharmaceutically acceptable salt thereof.

2. The method of claim 1, wherein the compound is further defined as: 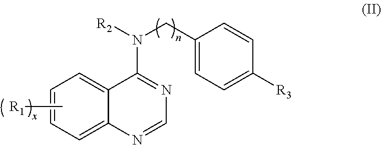
wherein: R₁, x, R₂, n, and R₃ are as defined above; or a pharmaceutically acceptable salt thereof.

3. The method of claim 1, wherein the compound is further defined as: 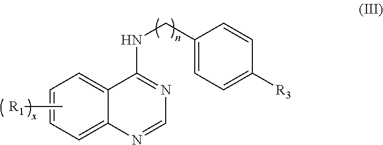
wherein: R₁, x, n, and R₃ are as defined above; or a pharmaceutically acceptable salt thereof.

4. The method according to claim 1, wherein the compound is further defined as: 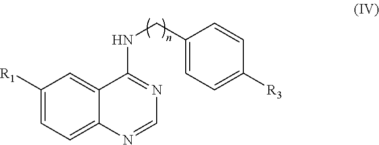
wherein: R₁, n, and R₃ are as defined above; or a pharmaceutically acceptable salt thereof.

5. The method according to claim 1, wherein R₁ is nitro.

6. The method according to claim 1, wherein R₁ is amino, alkylamino_{(C≤8)}, substituted alkylamino_{(C≤8)}, dialkylamino_{(C≤8)}, or substituted dialkylamino_{(C≤8)}.

7. The method according to claim 1, wherein n is 2 or 3.

8. (canceled)

9. The method according to claim 1, wherein R₃ is halo.

10. (canceled)

11. The method according to claim 1, wherein R₃ is amido_{(C≤8)} or substituted amido_{(C≤8)}.

12. (canceled)

13. The method according to claim 1, wherein the compound is further defined as: 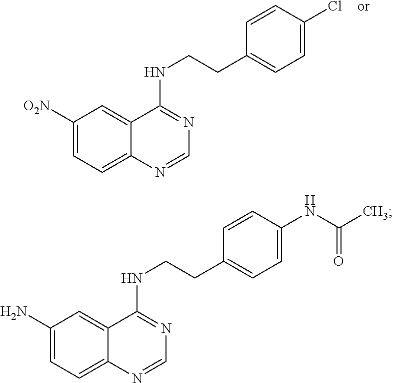 [SMILES: O=[N+]([O-])c1ccc2ncnc(NCCc3ccc(Cl)cc3)c2c1 ; CC(=O)Nc1ccc(CCNc2ncnc3ccc(N)cc23)cc1]
or a pharmaceutically acceptable salt thereof.

14. A method of treating a mammalian subject with Alzheimer's Disease comprising administering to said subject an agonist or TRPC6 or Orai2, wherein said agonist is not hyperforin or a hyperforin derivative.

15. A method of treating a mammalian subject with Alzheimer's Disease comprising administering to said subject an agonist of the nSOC pathway, wherein said agonist is not hyperforin or a hyperforin derivative or analog.

16. A method of treating a mammalian subject with Alzheimer's Dis comprising administering to said subject a potentiator of diacylglycerol (DAG)-induced TRPC6 activation.

17. The method of claim 1 wherein said subject is further treated with at least a second Alzheimer's Disease therapy.

18. (canceled)

19. The method of claim 1, wherein treating comprises one or more of improvements in memory, cognition or learning, slowing the progression of symptoms or pathophysiology, improving quality of life, or increasing life span.

20. The method of claim 1, wherein said compound or agonist is administered orally or by injection, including intravenously, intradermally, intraarterially, intraperitoneally, intracranially, intraarticularly, intraprostaticaly, intrapleurally, intramuscularly, or subcutaneously.

21. The method of claim 1, wherein said compound or agonist is administered 1, 2, 3 or 4 times daily.

22. The method of claim 1, wherein said compound or agonist is administered chronically.

23. The method of claim 1, further comprising measuring cognition or memory in said subject prior to and/or after administration of said compound or agonist.

24. (canceled)

25. The method of claim 1, wherein said human suffers from early onset Alzheimer's Disease.

26. The method of claim 1, wherein said human suffers from late onset Alzheimer's Disease.

27. (canceled)

28. A pharmaceutical composition comprising a compound of the formula: 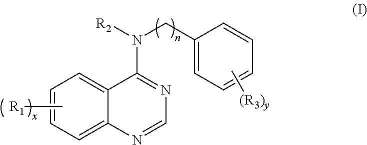
wherein:
each R₁ is independently selected from amino, cyano, carboxyl, halo, hydroxy, or nitro; or
alkylamino_{(C≤8)}, dialkylamino_{(C≤8)}, cycloalkylamino_{(C≤8)}, dicycloalkylamino_{(C≤8)}, or a substituted version of any of these groups
x is 1, 2, 3, 4, or 5;
R₂ is hydrogen, alkyl_{(C≤8)}, or substituted alkyl_{(C≤8)};
n is 1, 2, 3, 4, or 5;
each R₃ is independently selected from amino, carboxyl, cyano, halo, hydroxy, or nitro; or
alkyl_{(C≤8)}, cycloalkyl_{(C≤8)}, alkenyl_{(C≤8)}, alkynyl_{(C≤8)}, acyl_{(C≤8)}, amido_{(C≤8)}, or a substituted version of any of these groups; and
y is 1, 2, 3, 4, or 5;
or a pharmaceutically acceptable salt thereof formulated in a pharmaceutical buffer, diluent or excipient.

29-33. (canceled)
